(19) United States
(12) Patent Application Publication
(10) Pub. No.: US 2007/0254897 A1
(43) Pub. Date: Nov. 1, 2007

(54) Compositions and methods for the treatment of cardiovascular disease

(75) Inventor: Per Gjorstrup, Cambridge, MA (US)

Correspondence Address: FISH & NEAVE IP GROUP;ROPES & GRAY LLP, ONE INTERNATIONAL PLACE, BOSTON, MA, 02110-2624, US

(73) Assignee: Resolvyx Pharmaceuticals, Inc., Bedford, MA (US)

(21) Appl. No.: 11/796,611

(22) Filed: Apr. 27, 2007

Related U.S. Application Data
(60) Provisional application No. 60/796,070, filed on Apr. 28, 2006.
(60) Provisional application No. 60/831,831, filed on Jul. 19, 2006.

(51) Int. Cl. A61K 31/505; A61K 31/401; A61K 31/366; A61K 31/202; A61K 31/22

(52) U.S. Cl. 514/256; 514/423; 514/419; 514/460; 514/548; 514/560; 514/475

(57) ABSTRACT

The invention relates to methods of treating cardiovascular disease comprising administering a resolvin, lipoxin, or oxylipin compound.

### RELATED APPLICATIONS

This application claims priority to, and the benefit of, U.S. Provisional Applications Ser. Nos. 60/796,070, filed Apr. 28, 2006, and 60/831,831, filed Jul. 19, 2006, both of which are incorporated herein by reference.

### BACKGROUND

There are approximately 60 million people in the U.S. with risk factors for developing chronic cardiovascular diseases, including high blood pressure, stroke, diabetes, coronary artery disease, valvular heart disease, congenital heart disease, cardiomyopathy, and other disorders. Another 10 million patients have already suffered quantifiable structural heart damage but are presently asymptomatic.

In the United States, the complications of atherosclerosis account for about one half of all deaths and for about one third of deaths in persons between 35 and 65 years of age. Atherosclerosis, or the developments of atheromatous plaques in large and medium-sized arteries, is the most common form of arteriosclerosis. Many factors are associated with the acceleration of atherosclerosis, regardless of the underlying primary pathogenic change, for example, age, elevated plasma cholesterol level, high arterial blood pressure, cigarette smoking, reduced high-density lipoprotein (HDL) cholesterol level, or family history of premature coronary artery disease.

The risk of death from coronary artery disease has a continuous and graded relation to total serum cholesterol levels greater than 180 mg/dl (Stamler et al., JAMA, Volume 256, 2823, 1986). Approximately one third of adults in the United States have levels that exceed 240 mg/dl and, therefore, have a risk of coronary artery disease that is twice that of people with cholesterol levels lower than 180 mg/dl. Acceleration of atherosclerosis is principally correlated with elevation of LDL, or beta fraction, which is rich in cholesterol but poor in triglycerides. Elevation of HDL or alpha fraction, has a negative correlation with atherosclerosis (Castelli et al., JAMA, Volume 256, 2835, 1986). HDL exerts a protective effect and the ratio of total cholesterol to HDL cholesterol is a better predictor of coronary artery disease than the level of either alone. Total cholesterol levels are classified as being desirable (<200 mg/dl), borderline (200-239 mg/dl), or high (>240 mg/di) (Report of the National Education Expert Panel on Detection, Evaluation, and Treatment of High Blood Cholesterol in Adults, Arch. Intern. Med., Volume 148, 36, 1988).

Advances in the study of cholesterol metabolism and coronary disease have initiated an era of increased emphasis on preventive therapy. New guidelines for the detection and treatment of high blood cholesterol in adults recommend that patients with high cholesterol levels or with borderline-high levels and two or more additional risk factors should have a measurement of LDL. LDL cholesterol levels are then classified as borderline-high risk (130-159 mg/dl) or high risk (>160 mg/dl). Dietary treatment is recommended for those patients with high-risk levels who have two or more additional risk factors. Drug treatment is recommended for all patients with LDL levels greater than 189 mg/dl and for those patients with LDL cholesterol levels between 159 and 189 mg/dl who have two or more additional risk factors.

Despite advances, there remains a need for improved treatment of cardiovascular diseases and to increase (or prevent the decrease of) HDL and HDL/LDL ratios.

### SUMMARY OF INVENTION

The present invention provides methods for the treatment of cardiovascular disease in a patient comprising administering to the patient a a compound of formula A, a compound of any one of formulae 1 to 44, a lipoxin compound, or an oxylipin compound, or a combination of aspirin and an omega-3 fatty acid conjointly with a statin.

The present invention provides a method for increasing or preventing the decrease of serum HDL concentration and for decreasing or preventing the increase of serum LDL/HDL ratio in a patient. These methods comprise administering to a patient a pharmaceutically acceptable composition comprising a compound of formula A, a compound of any one of formulae 1 to 44, a lipoxin compound, or an oxylipin compound, or a combination of aspirin and an omega-3 fatty acid. These methods may additionally comprise administering to the patient a statin, either as a separate dosage form or as part of the compound of formula A, compound of any one of formulae 1 to 44, lipoxin compound, oxylipin compound, or aspirin and/or omega-3 fatty acid composition.

The invention also provides a method of decreasing the dose of a statin required to achieve a desired increase in serum HDL, or serum HDL/LDL ratio, or a decrease in serum total cholesterol level in a patient comprising administering to a patient a compound of formula A, a compound of any one of formulae 1 to 44, a lipoxin compound, or an oxylipin compound, or a combination of aspirin and an omega-3 fatty acid conjointly with said statin.

The present invention also provides pharmaceutically acceptable compositions comprising a statin and a compound of formula A, a compound of any one of formulae 1 to 44, a lipoxin compound, or an oxylipin compound disclosed herein, or a combination of aspirin and an omega-3 fatty acid.

### DETAILED DESCRIPTION OF THE INVENTION

The present invention provides a method of treating cardiovascular disease in a patient comprising administering to said patient a compound of formula A, a compound of any one of formulae 1 to 44, a lipoxin compound, or an oxylipin compound, or a combination of aspirin and an omega-3 fatty acid conjointly with a statin.

Compounds of formula A, compounds of any one of formulae 1 to 44, lipoxin compounds, and oxylipin compounds are capable of resolving inflammation. The combination of aspirin and an omega-3 fatty acid produces active metabolites that are also capable of resolving inflammation. Several aspects of cardiovascular disease, in particular the formation of atherosclerotic vessel wall plaques, are believed to be intimately related to inflammation. Today it is believed that serum markers of inflammation such as CRP may be as predictive of risk of cardiovascular disease as elevated levels of LDL. Thus, compounds of formula A, compounds of any one of formulae 1 to 44, lipoxin compounds, oxylipin compounds, or a combination of aspirin and an omega-3 fatty acid have been suggested as being useful to treat cardiovascular disease.

One mechanism by which compounds of formula A, compounds of any one of formulae 1 to 44, lipoxin compounds, oxylipin compounds, or a combination of aspirin and an omega-3 fatty acid may be effective in treating cardiovascular disease is by inhibiting the structural and functional modifications of HDL that are an immediate effect of the acute phase response commonly seen in cardiovascular disease with active atherosclerotic vessel wall plaques. Thus, compounds of formula A, compounds of any one of formulae 1 to 44, lipoxin compounds, oxylipin compounds, or a combination of aspirin and an omega-3 fatty acid can increase HDL levels (or prevent the decrease of HDL levels) and restore the LDL scavenging effects of HDL. This leads to a lower and improved serum LDL/HDL ratio.

In addition to increasing HDL levels, statins also demonstrate anti-inflammatory activity which contributes to their ability to lower cardiovascular disease risk and treat cardiovascular disease. However, the full anti-inflammatory potential of statins cannot be utilized clinically as a monotherapy due to the high doses required, which can lead to an increased rate and severity level of treatment-limiting adverse events, notably liver toxicity.

Advantageously and surprisingly, treatment of cardiovascular disease with a combination of a statin and a compound of formula A, a compound of any one of formulae 1 to 44, a lipoxin compound, an oxylipin compound, or a combination of aspirin and an omega-3 fatty acid leads to a mutual enhancement of both the anti-inflammatory properties and the serum HDL elevating properties of the two classes of compounds while avoiding the risks associated with high doses of statins alone.

Cardiovascular disease refers to one or more disease states of the cardiovascular tree (including the heart). Diseases of the cardiovascular tree and diseases of dependent organs include, for example, but are not limited to any one or more of:

disorders of the heart muscle (cardiomyopathy or myocarditis) such as idiopathic cardiomyopathy, metabolic cardiomyopathy which includes diabetic cardiomyopathy, alcoholic cardiomyopathy, drug-induced cardiomyopathy, ischemic cardiomyopathy, and hypertensive cardiomyopathy;

atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries;

toxic, drug-induced, and metabolic (including hypertensive and/or diabetic) disorders of small blood vessels (microvascular disease) such as the retinal arterioles, the glomerular arterioles, the vasa nervorum, cardiac arterioles, and associated capillary beds of the eye, the kidney, the heart, and the central and peripheral nervous systems; and,

plaque rupture of atheromatous lesions of major blood vessels such as the aorta, the coronary arteries, the carotid arteries, the cerebrovascular arteries, the renal arteries, the iliac arteries, the fermoral arteries and the popliteal arteries.

Yet other disorders that may be treated with compounds of the invention include restenosis, e.g., following coronary intervention, and disorders relating to an abnormal level of high density and low density cholesterol.

In methods of the invention, wherein a compound of formula A, a compound of any one of formulae 1 to 44, a lipoxin compound, or an oxylipin compound, or a combination of aspirin and an omega-3 fatty acid is administered conjointly with a statin, the statin may be chosen from any statin known in the art. Statins suitable for said conjoint administration include, but are not limited to, mevastatin ((2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), atorvastatin ((βR,δR)-2-(4-fluorophenyl)-β,δ-dihydroxy-5-(1-methylethyl)-3-phenyl-4-[(phenylamino)carbonyl]-1H-Pyrrole-1-heptanoic acid), fluvastatin ((3R,5S,6E)-rel-7-[3-(4-fluorophenyl)-1-(1-methylethyl)-1H-indol-2-yl]-3,5-dihydroxy-6-heptenoic acid), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin ((βR,δR,1S,2S,6S,8S,8aR)-1,2,6,7,8,8a-hexahydro-β,β,6-trihydroxy-2-methyl-8-[(2S)-2-methyl-1-oxobutoxy]-1-naphthaleneheptanoic acid), simvastatin (2,2-dimethyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), rosuvastatin ((3R,5 S,6E)-7-[4-(4-fluorophenyl)-6-(1-methylethyl)-2-[methyl(methylsulfonyl)amino]-5-pyrimidinyl]-3,5-dihydroxy-6-heptenoic acid), eptastatin, pitavastatin ((3R,5S,6E)-7-[2-cyclopropyl-4-(4-fluorophenyl)-3-quinolinyl]-3,5-dihydroxy-6-heptenoic acid), cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid), berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-, 2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-;4-(4-fluorophenyl)-2-(1-methylethyl)-3-[2-(tetrahydro-4-hydroxy-6-oxo-2H-pyran-2-yl)ethenyl]-1(2H)-isoquinolinone), SDZ-265859, BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5 S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,4R)-tetrahydro-4-hydroxy-6-oxo-2H-pyran-2-yl]ethyl]-1-naphthalenyl ester), and L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester).

For example, statins suitable for use in the methods of this invention include statins of formula 200: 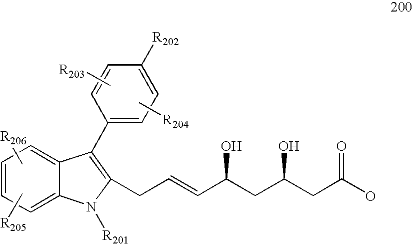
wherein
R₂₀₁ is selected from alkyl, alkenyl, alkynyl, cycloalkyl or aralkyl;
R₂₀₂, R₂₀₃ and R₂₀₄ are independently selected from hydrogen, halogen, alkyl, alkenyl or alkynyl; and
R₂₀₅ and R₂₀₆ are independently selected from hydrogen, halogen, alkyl, alkenyl, alkynyl, cycloalkyl, aralkyl, alkoxy or aralkoxy;
   or enantiomers or salts or hydrates thereof.

Other statins suitable for use in the methods of this invention include statins of formula 201:
A-B
wherein
A is selected from 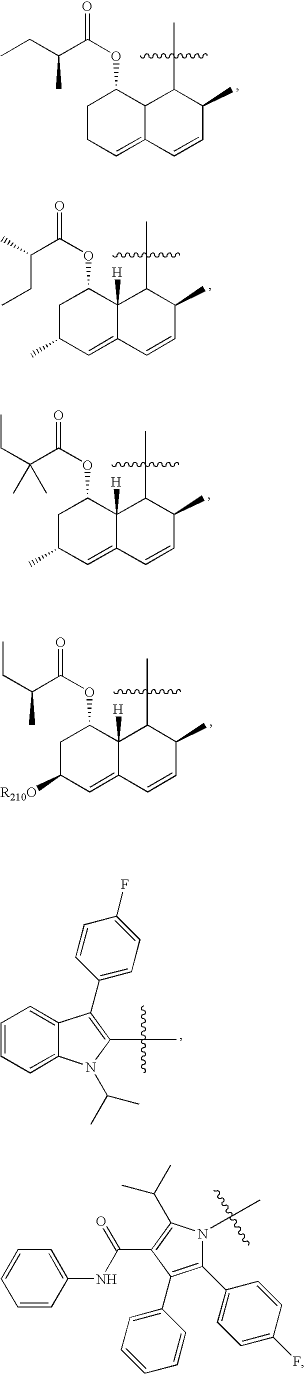 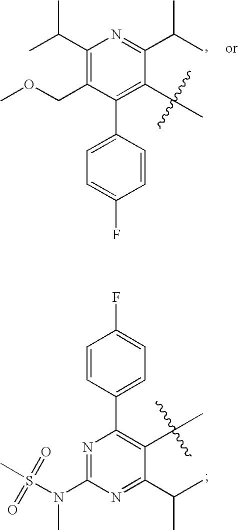
B is selected from 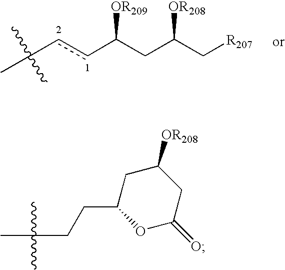
C1 and C2 are joined by a single or a double bond;
R₂₀₇ is selected from CO₂R₂₁₅, CONR₂₁₁R₂₁₂ or CH₂OR₂₁₃, or R₂₀₇ and R₂₀₉ can form a lactone;
R₂₁₅ is selected from H or a cationic salt moiety, or CO₂R₂₁₅ forms a pharmaceutically acceptable ester moiety;
R₂₀₈, R₂₀₉ and R₂₁₀ are independently selected from H, C(O)R₂₁₄ or C(O)NR₂₁₁R₂₁₂;
R₂₁₁ and R₂₁₂ are independently selected from H, alkyl, alkenyl or alkynyl;
R₂₁₃ is selected from H or C(O)R₂₁₄; and
R₂₁₄ is selected from alkyl, alkenyl or alkynyl.

Other statins suitable for use in the methods of this invention include statins of formula 202: 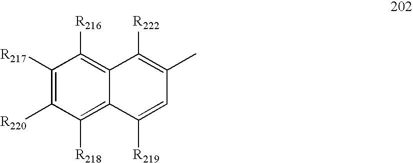
wherein
R₂₂₂ is selected from 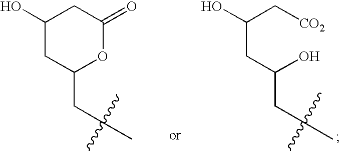
R₂₁₆ is selected from OH, C₆H₅CO₂ or R₂₂₁CO₂;
R₂₂₁ is a branched or straight C₁-C₅ alkyl, C₂-C₅ alkenyl, or C₂-C₅ alkynyl;
R₂₁₇, R₂₁₈ and R₂₁₉ are independently selected from H, C₁-C₅ alkyl, C₂-C₅ alkenyl, C₂-C₈ alkynyl or C₁-C₅ acyl; and
R₂₂₀ is selected from H or CH₃.

Other statins suitable for use in the methods of this invention include statins of formula 203: 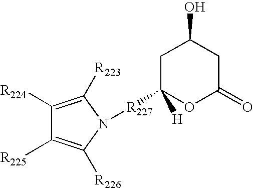
wherein
R₂₂₇ is —CH₂—, —CH₂—CH₂—, —CH₂—CH₂—CH₂— or —CH₂—CH(CH₃)—;
R₂₂₃ is 1-naphthyl; 2-naphthyl; cyclohexyl; norbornenyl; 2-, 3-, or 4-pyridinyl; phenyl, phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms;
Either R₂₂₄ or R₂₂₅ is —CONR₂₂₈R₂₂₉ where R₂₂₈ and R₂₂₉ are independently hydrogen; alkyl, alkenyl, or alkynyl of from one to six carbon atoms; 2-, 3-, or 4-pyridinyl; phenyl; phenyl substituted with fluorine, chlorine, bromine, cyano, trifluoromethyl, or carboalkoxy of from three to eight carbon atoms; and the other of R₂₂₄ or R₂₂₅ is hydrogen; alkyl, alkenyl, or alkynyl of from one to six carbon atoms; cyclopropyl; cyclobutyl; cyclopentyl; cyclohexyl; phenyl; or phenyl substituted with fluorine, chlorine, bromine, hydroxyl; trifluoromethyl; alkyl, alkenyl, or alkynyl of from one to four carbon atoms, alkoxy of from one to four carbon atoms, or alkanoyloxy of from two to eight carbon atoms; and
R₂₂₆ is alkyl, alkenyl, or alkynyl of from one to six carbon atoms; cyclopropyl; cyclobutyl; cyclopentyl; cyclohexyl; or trifluoromethyl;
or the hydroxyl acids, and pharmaceutically acceptable salts thereof, derived from the opening of the lactone ring.

Other statins suitable for use in the methods of this invention include statins of formula 204: 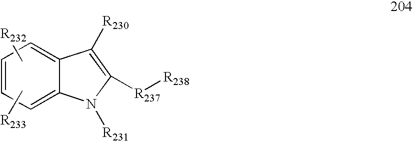
wherein
one of R₂₃₀ and R₂₃₁ is 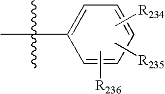
   and the other is primary or secondary C₁₋₆ alkyl, alkenyl, or alkynyl not containing an asymmetric carbon atom, C₃₋₆ cycloalkyl or phenyl-(CH₂)ₘ—;
R₂₃₄ is selected from hydrogen, C₁₋₃ alkyl, C₂-C₄ alkenyl, C₂-C₄ alkynyl, n-butyl, i-butyl, t-butyl, C₁₋₃ alkoxy, n-butoxy, i-butoxy, trifluoromethyl, fluoro, chloro, phenoxy or benzyloxy;
R₂₃₅ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy;
R₂₃₆ is selected from hydrogen, C₁₋₂ alkyl, C₂ alkenyl, C₂ alkynyl, C₁₋₂ alkoxy, fluoro or chloro;
m is selected from 1, 2 or 3, with the provisos that both R₂₃₅ and R₂₃₆ must be hydrogen when R₂₃₄ is hydrogen, R₂₃₆ must be hydrogen when R₂₃₅ is hydrogen, not more than one of R₂₃₄ and R₂₃₅ is trifluoromethyl, not more than one of R₂₃₄ and R₂₃₅ is phenoxy, and not more than one of R₂₃₄ and R₂₃₅ is benzyloxy;
R₂₃₂ is selected from hydrogen, C₁₋₃ alkyl, C₂-C₄ alkenyl, C₂-C₄ alkynyl, n-butyl, i-butyl, t-butyl, C₃₋₆ cycloalkyl, C₁₋₃ alkoxy, n-butoxy, i-butoxy, trifluoromethyl, fluoro, chloro, phenoxy or benzyloxy;
R₂₃₃ is selected from hydrogen, C₁₋₃ alkyl, C₂-C₃ alkenyl, C₂-C₃ alkynyl, C₁₋₃ alkoxy, trifluoromethyl, fluoro, chloro, phenoxy or benzyloxy, with the provisos that R₂₃₃ must be hydrogen when R₂₃₂ is hydrogen, not more than one of R₂₃₂ and R₂₃₃ is trifluoromethyl, not more than one of R₂₃₂ and R₂₃₃ is phenoxy, and not more than one of R₂₃₂ and R₂₃₃ is benzyloxy;
R₂₃₇ is selected from —(CH₂)ₙ— or —CH═CH—, wherein n is 0, 1, 2 or 3;
R₂₃₈ is selected from 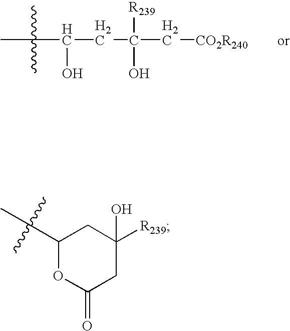
R₂₃₉ is selected from hydrogen, or C₁₋₃ alkyl, C₂-C₃ alkenyl, or C₂-C₃ alkynyl;
R₂₄₀ is selected from hydrogen, R₂₄₁ or M;
R₂₄₁ is a physiologically acceptable and hydrolyzable ester group; and
M is a pharmaceutically acceptable cation.

Other statins suitable for use in the methods of this invention include statins of formula 205: 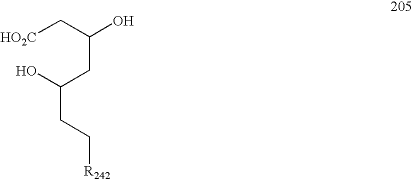
wherein
R₂₄₂ is selected from 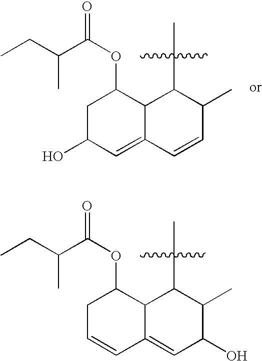
   or ring-closed lactones, salts or esters thereof.

Other statins suitable for use in the methods of this invention include statins of formula 206: 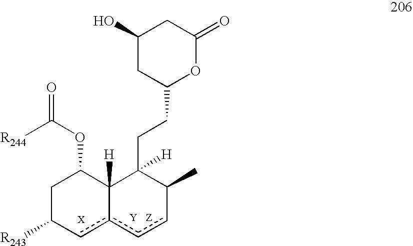
wherein
R₂₄₃ is selected from H or CH₃;
R₂₄₄ is selected from 1,1-dimethylpropyl; C₃₋₁₀cycloalkyl; C₂₋₁₀alkenyl; C₁₋₁₀CF₃-substituted alkyl; phenyl; halophenyl; phenyl-C₁₋₃alkyl; substituted phenyl-C₁₋₃alkyl in which the substituent is halo, C₁₋₃alkyl or C₁₋₃alkoxy;
the dotted lines at X, Y and Z represent possible double bonds, said double bonds, when any are present, being either X and Z in combination or X, Y or Z alone;
or the corresponding dihydroxy acid of formula 206a 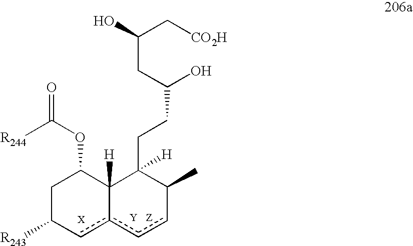
or a pharmaceutically acceptable salt of said acid, a C₁₋₄alkyl ester of said acid or a phenyldimethylamino-, or acetylamino-substituted-C₁₋₄alkyl ester of said acid.

Other statins suitable for use in the methods of this invention include statins of formula 207:

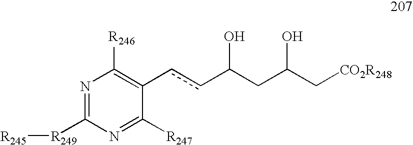

wherein
R₂₄₅ is lower alkyl, alkenyl, alkynyl, aryl or aralkyl, each of which may have one or more substituents;
R₂₄₆ and R₂₄₇ independently are selected from hydrogen, lower alkyl, alkenyl, alkynyl, or aryl, and each of said lower alkyl, alkenyl, alkynyl and aryl may have one or more substituents;
R₂₄₈ is hydrogen, lower alkyl, alkenyl, alkynyl, or a cation capable of forming a non-toxic pharmaceutically acceptable salt;
R₂₄₉ is sulfur, oxygen, or sulfonyl, or imino which may have a substituent; and the dotted line represents the presence or absence of a double bond;
   or the corresponding ring-closed lactone.

Compounds suitable for use in methods of the invention include those of Formula A, 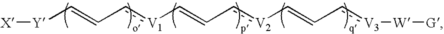
wherein:

each of W′ and Y′ is a bond or a linker independently selected from a ring containing up to 20 atoms or a chain of up to 20 atoms, provided that W′ and Y′ can independently include one or more nitrogen, oxygen, sulfur or phosphorous atoms, further provided that W′ and Y′ can independently include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, or sulfonyl, further provided that W′ and Y′ can independently contain one or more fused carbocyclic, heterocyclic, aryl or heteroaryl rings, and further provided that when o′ is 0, and V₁ is 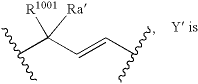
connected to V₁ via a carbon atom;

V₁ is selected from 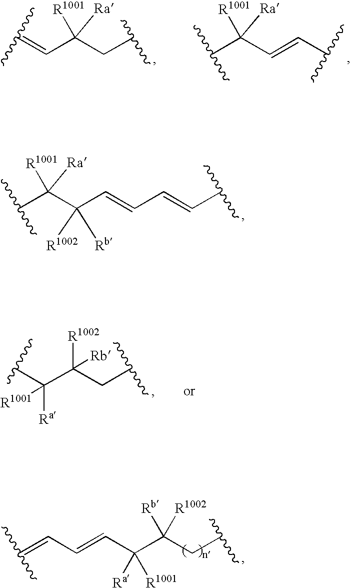
wherein when q′ is 0 and V₃ is a bond, n′ is 0 or 1; otherwise n′ is 1;

V₂ is selected from a bond, 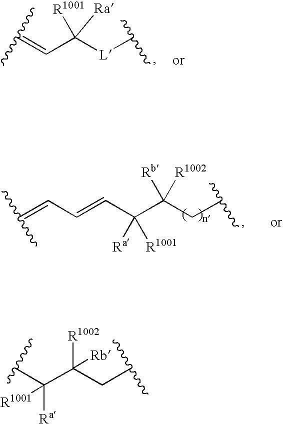
wherein:
L′ is selected from —C(R¹⁰⁰³)(R¹⁰⁰⁴)—, wherein each of R¹⁰⁰³ and R¹⁰⁰⁴ is independently selected from hydrogen, alkyl, alkenyl, alkynyl, perfluoroalkyl, alkoxy, aryl or heteroaryl, or R¹⁰⁰³ and R¹⁰⁰⁴ are connected together to form a carbocyclic or heterocyclic ring; when V₃ is 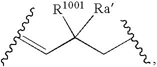
   L′ is additionally selected from W′; and n′ is 0 or 1;

V₃ is selected from a bond or 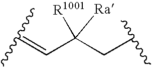
wherein:
each R¹⁰⁰¹ and R¹⁰⁰² is independently for each occurrence selected from hydrogen, alkyl, alkenyl, alkynyl, aryl, heteroaryl, alkylaryl, alkoxy, or halo, wherein said alkyl- or aryl-containing moiety is optionally substituted with up to 3 independently selected substituents;
each of R^{a′} and R^{b′} is independently for each occurrence selected from —OR′ or —N(R′)₂, or adjacent R^{a′} and R^{b′} are taken together to form an epoxide ring having a cis or trans configuration, wherein each R′ is independently selected from hydrogen, alkyl, alkenyl, alkynyl, aryl, heteroaryl, acyl, silyl, alkoxyacyl, aminoacyl, aminocarbonyl, alkoxycarbonyl, or a protecting group;

or when V₁ is 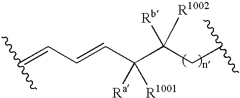
and V₂ is 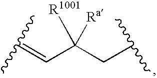
R¹⁰⁰² and R^{b′} are both hydrogen;

X′ is selected from —CN, —C(NH)N(R″)(R″), —C(S)-A′, —C(S)R″, —C(O)-A′, —C(O)—R″, —C(O)—SR″, —C(O)—NH—S(O)₂—R″, —S(O)₂-A′, —S(O)₂—R″, S(O)₂N(R″)(R″), —P(O)₂-A′, —PO(OR″)-A′, -tetrazole, alkyltetrazole, or —CH₂OH, wherein
A′ is selected from —OR″, —N(R″)(R″) or —OM′;
each R″ is independently selected from hydrogen, alkyl, aryl, arylalkyl, heteroaryl, heteroarylalkyl or a detectable label molecule, wherein any alkyl-, aryl- or heteroaryl-containing moiety is optionally substituted with up to 3 independently selected substituents; and
M′ is a cation;

G′ is selected from hydrogen, halo, hydroxy, alkyl, aryl, arylalkyl, heteroaryl, heteroarylalkyl, alkoxy, aryloxy, carboxy, amino, alkylamino, dialkylamino, acylamino, carboxamido or a detectable label molecule, wherein any alkyl-, aryl- or heteroaryl-containing moiety is optionally substituted with up to 3 independently selected substituents;

o′ is 0, 1, 2, 3, 4, or 5;

p′ is 0, 1, 2, 3, 4, or 5;

q′ is 0, 1, or 2; and

o′+p′+q′ is 1, 2, 3, 4, 5 or 6;

wherein:

if V₂ is a bond, then q′ is 0, and V₃ is a bond;

if V₃ is 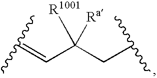
then o′ is 0, V₁ is 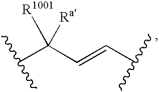
p′ is 1 and V₂ is

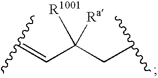

any acyclic double bond may be in a cis or a trans configuration or is optionally replaced by a triple bond; and

either one 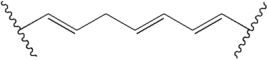
portion of the compound, if present, is optionally replaced by 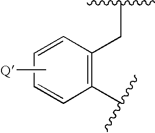
or one 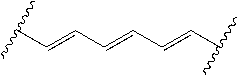
portion of the compound, if present, is optionally replaced by 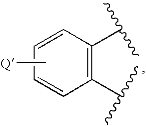
wherein Q′ represents one or more substituents and each Q′ is independently selected from halo, alkyl, alkenyl, alkynyl, cycloalkyl, aryl, heteroaryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl.

In certain embodiments, V₁ is selected from

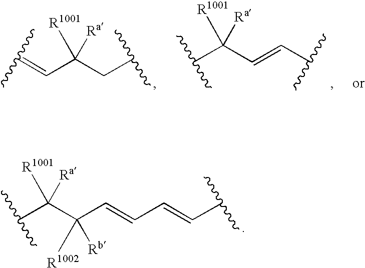

In certain embodiments, V₂ is selected from a bond,

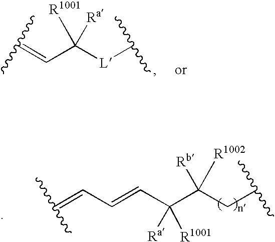

In certain embodiments, when q′ is 0 and V₃ is a bond, n′ is 0 or 1; otherwise n′ is 1.

In certain embodiments, p′ is 0, 1, 2, 3, or 5.

In certain embodiments, q′ is 0 or 1.

In certain embodiments, if V₁ 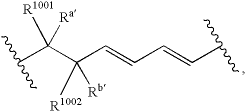
then o′ is 0 or 1, p′ is 1 or 2, o′+p′ is or 2, V₂ is 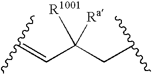
and V₃ is a bond.

In certain embodiments, if V₁ is 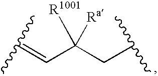
then o'is 3, 4 or 5, p′ is 0, 1 or 2, o′+p′ is 4 or 5, and V₂ is a bond.

In certain embodiments, if V₂ is a bond, then o′ is 0, 3, 4 or 5; p′ is 0, 1, 2 or 5, o′+p′ is 4 or 5, q′ is 0, and V₃ is a bond.

In certain embodiments, each of W′ and Y′ is independently selected from a bond or lower alkyl or heteroalkyl optionally substituted with one or more substituents independently selected from alkenyl, alkynyl, aryl, chloro, iodo, bromo, fluoro, hydroxy, amino, or oxo.

Compounds suitable for use in methods of the invention include those of Formula 1, 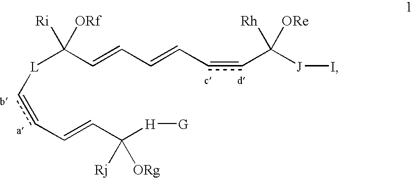
wherein
Carbons a′ and b′ are connected by a double bond or a triple bond;
Carbons c′ and d′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
Rh, Ri and Rj are independently selected from hydrogen, alkyl, alkenyl, alkynyl, perfluoroalkyl, aryl or heteroaryl;
I is selected from —C(O)-E, —SO₂-E, —PO(OR)-E, where E is hydroxy, alkoxy, aryloxy, amino, alkylamino, dialkylamino, or arylamino; and R is hydroxyl or alkoxy;
J, L and H are linkers independently selected from a ring containing up to 20 atoms or a chain of up to 20 atoms, provided that J, L and H can independently include one or more nitrogen, oxygen, sulfur or phosphorous atoms, and further provided that J, L and H can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J, L and H can also contain one or more fused carbocyclic, heterocyclic, aryl or heteroaryl rings, and provided that linker J is connected to the adjacent C(R)OR group via a carbon atom;
G is selected from hydrogen, alkyl, perfluoroalkyl, alkenyl, alkynyl, aryl, heteroaryl, chloro, iodo, bromo, fluoro, hydroxy, alkoxy, aryloxy, carboxy, amino, alkylamino, dialkylamino, acylamino, or carboxamido;
   or pharmaceutically acceptable salts thereof.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

In certain embodiments, a compound of formula 1 is represented by formula 2, 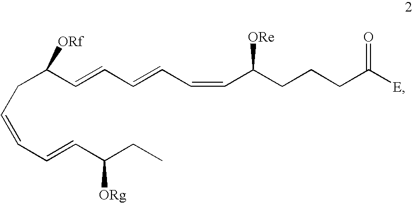
wherein
E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 2 include:

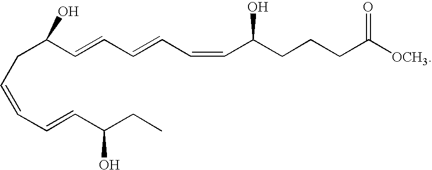

In certain embodiments, a compound of formula 1 is represented by formula 3, 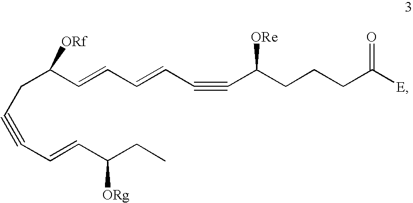
wherein
E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 3 include:

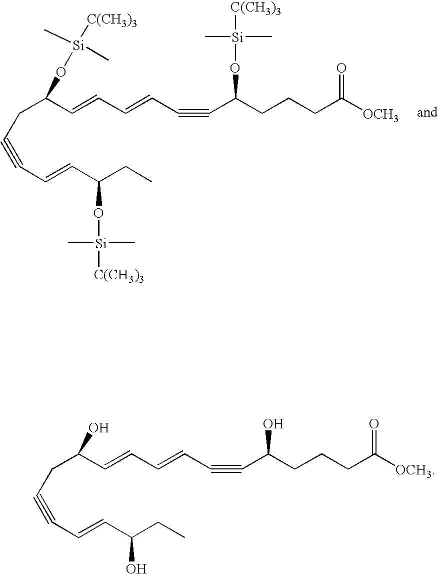

Other compounds suitable for use in methods of the invention include those of Formula 4, 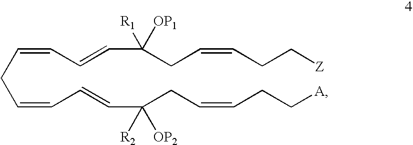
wherein
A is H or —OP₄;
P₁, P₂ and P₄ each individually is a protecting group or hydrogen atom;
R₁ and R₂ each individually is a substituted or unsubstituted, branched or unbranched alkyl, alkenyl, or alkynyl group, substituted or unsubstituted aryl group, substituted or unsubstituted, branched or unbranched alkylaryl group, halogen atom, hydrogen atom;
Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN, preferably a carboxylic acid, ester, amide, thioester, thiocarboxamide or a nitrile;
each R^{a}, if present, is independently selected from hydrogen, (C1-C6) alkyl, (C2-C6) alkenyl, (C2-C6) alkynyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered heterocyclyl, morpholinyl, piperazinyl, homopiperazinyl, piperidinyl, 4-11 membered heterocyclylalkyl, 5-10 membered heteroaryl and 6-16 membered heteroarylalkyl;
each R^{b}, if present, is a suitable group independently selected from ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, two R^{c} taken together with the nitrogen atom to they are bonded form a 5 to 8-membered heterocyclyl or heteroaryl which optionally including one or more additional heteroatoms and optionally substituted with one or more of the same or different R^{a} or suitable R^{b} groups;
each n independently is an integer from 0 to 3;
each R^{d} independently is a protecting group or R^{a};
   or pharmaceutically acceptable salts thereof.

Other compounds suitable for use in methods of the invention include those of Formula 5, 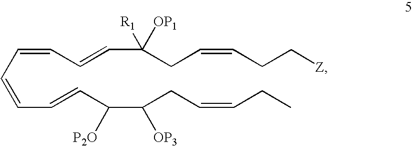
or pharmaceutically acceptable salts thereof, wherein
P₃ is a protecting group or hydrogen atom; and
P₁, P₂, R₁ and Z are as defined above in formula 4.

Other compounds suitable for use in methods of the invention include those of Formula 6, 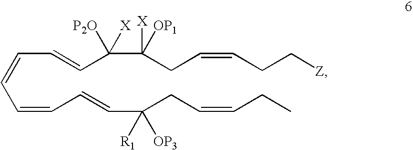
or pharmaceutically acceptable salts thereof, wherein
each X represents hydrogen or taken together both X groups represent one substituted or unsubstituted methylene, an oxygen atom, a substituted or unsubstituted N atom, or a sulfur atom such that a three-membered ring is formed; and
P₁, P₂, P₃, R₁ and Z are as defined above.

Other compounds suitable for use in methods of the invention include those of Formula 7, 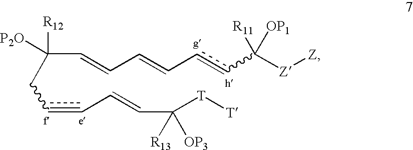
or pharmaceutically acceptable salts thereof, wherein
Carbons e′ and f′ are connected by a double bond or a triple bond, and when carbon e′ is connected to carbon f′ through a double bond the stereochemistry is cis or trans;
Carbons g′ and h′ are connected by a double bond or a triple bond and when carbon g′ is connected to carbon h′ through a double bond the stereochemistry is cis or trans;
m is 0 or 1;
T′ is hydrogen, (C1-C6) alkyl, (C2-C6) alkenyl, (C2-C6) alkynyl, (C5-C14) aryl, (C6-C16) arylalkyl, 5-14 membered heteroaryl, 6-16 membered heteroarylalkyl, or —CH═CHCH₂CH₃;
T is —(CH₂)_{q}— or —(CH₂)_{q}—O—, where q is an integer from 0 to 6;
Z′ is (C1-C6) alkylene optionally substituted with 1, 2, 3, 4, 5 or 6 of the same or different halogen atoms, —(CH₂)ₚ—O—CH₂— or —(CH₂)ₘ—S—CH₂—, where p is an integer from 0 to 4;
R₁₁, R₁₂ and R₁₃ each individually is substituted or unsubstituted, branched or unbranched alkyl, alkenyl, or alkynyl group, substituted or unsubstituted aryl group, substituted or unsubstituted, branched or unbranched alkylaryl group, C₁₋₄alkoxy, halogen atom, —CH₂R₁₄, —CHR₁₄R₁₄, —CR₁₄R₁₄R₁₄, or a hydrogen atom;
R₁₄ is independently for each occurrence selected from —CN, —NO₂ or halogen;
P₁, P₂, P₃, and Z are as defined above.

Other compounds suitable for use in methods of the invention include those of Formula 8, 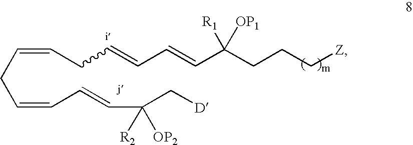
or pharmaceutically acceptable salts thereof, wherein
the stereochemistry of the carbon i′ to carbon j′ bond is cis or trans;
m is 0 or 1;
D′ is CH₃, —CH═CHCH₂U or —CH═CHCH₂CH₂A;
U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
A is H or —OP₄;
P₁, P₂, P₄, R₁, R₂ and Z are as defined above.

Other compounds suitable for use in methods of the invention include those of Formula 9, 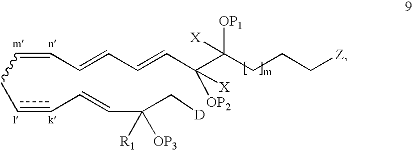
or pharmaceutically acceptable salts thereof, wherein
Carbons k′ and l′ are connected by a double bond or a triple bond;
the stereochemistry of the carbon m′ to carbon n′ double bond is cis or trans;
m is 0 or 1;
D is —CH₃ or —CH═CHCH₂CH₃;
P₁, P₂, P₃, R₁, X, and Z are as defined above.

Other compounds suitable for use in methods of the invention include those of Formula 10, 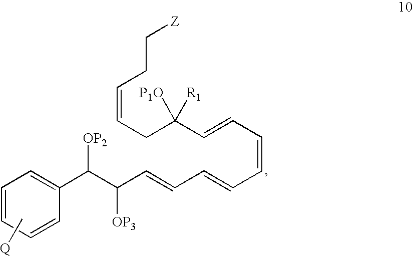
or pharmaceutically acceptable salts thereof, wherein
P₁, P₂, P₃, R₁ and Z are as defined above; and
Q represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group.

Other compounds suitable for use in methods of the invention include those of Formula 11, 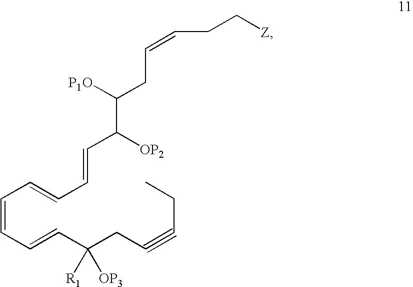
or pharmaceutically acceptable salts thereof, wherein
P₁, P₂, P₃, R₁, and Z are as defined above.

Other compounds suitable for use in methods of the invention include those of Formula 12, 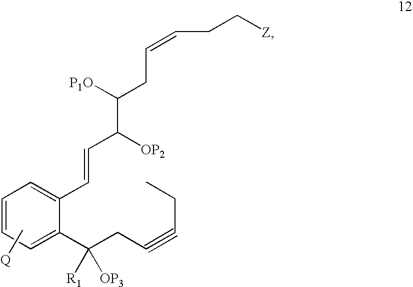
or pharmaceutically acceptable salts thereof, wherein
P₁, P₂, P₃, Q, R₁, and Z are as defined above.

Other compounds suitable for use in methods of the invention include those of Formula 13, 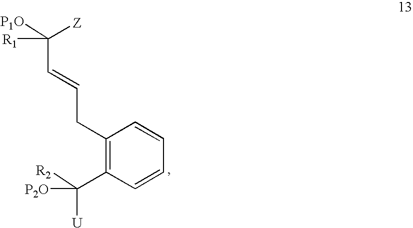
or pharmaceutically acceptable salts thereof, wherein
P₁, P₂, R₁, R₂, U, and Z are as defined above.

Other compounds suitable for use in methods of the invention include those of Formula 14, 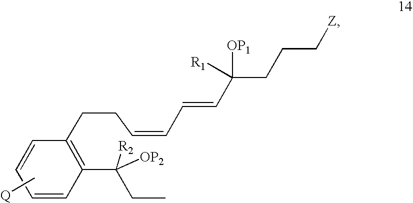
or pharmaceutically acceptable salts thereof, wherein
P₁, P₂, R₁, R₂, Q, and Z are as defined above.

Other compounds suitable for use in methods of the invention include those of Formula 15, 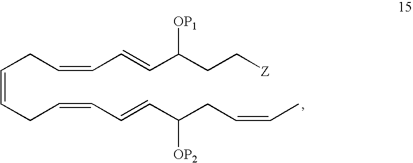
or pharmaceutically acceptable salts thereof, wherein
P₁, P₂, and Z are as defined above.

Other compounds suitable for use in methods of the invention include those of Formula 16, 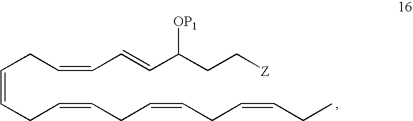
or pharmaceutically acceptable salts thereof, wherein
P₁ and Z are as defined above.

Other compounds suitable for use in methods of the invention include those of Formula 17, 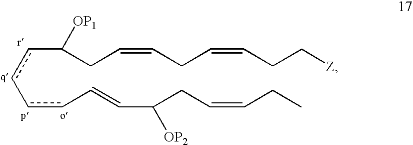
or pharmaceutically acceptable salts thereof, wherein
Carbons o′ and p′ are connected by a single or a double bond;
Carbons q′ and r′ are connected by a single or a double bond; and
P₁, P₂, and Z are as defined above.

Other compounds suitable for use in methods of the invention include those of Formula 18, 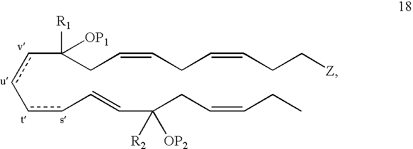
or pharmaceutically acceptable salts thereof, wherein
the stereochemistry of the carbon s′ to carbon t′ double bond is cis or trans;
the stereochemistry of the carbon u′ to carbon v′ double bond is cis or trans; and
P₁, P₂, R₁, R₂, and Z are as defined above.

Other compounds suitable for use in methods of the invention include those of Formula 19, 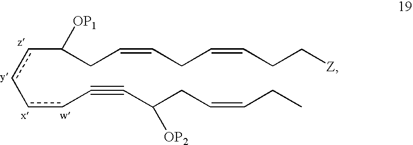
or pharmaceutically acceptable salts thereof, wherein
Carbons w′ and x′ are connected by a single or a double bond;
Carbons y′ and z′ are connected by a single or a double bond; and
P₁, P₂, and Z are as defined above.

In certain embodiments of formulae 4 to 19, each R^{b}, if present, is a suitable group independently selected from ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c}.

Other compounds suitable for use in methods of the invention include those of Formula 20, 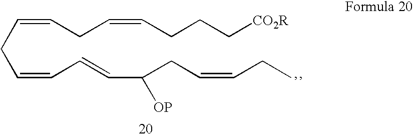 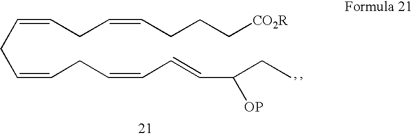 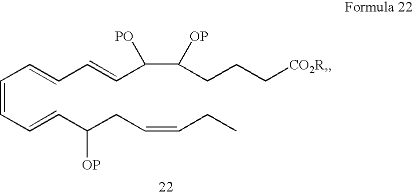 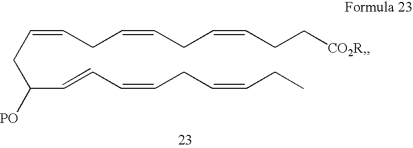 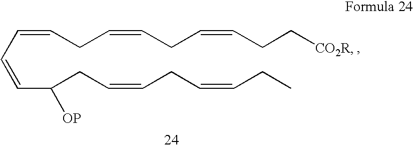 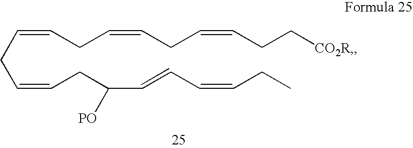 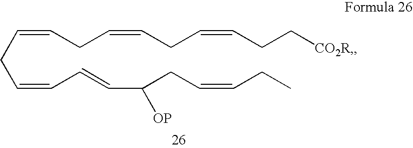 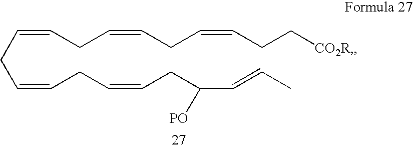 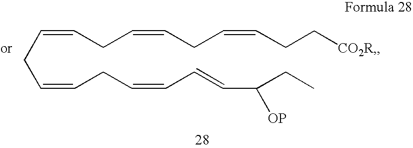
or pharmaceutically acceptable salts of any of the above, wherein
each P is individually selected from H or a protecting group; and
R is H, C₁₋₆alkyl (e.g., methyl, ethyl, glycerol), C₂₋₆alkenyl or C₂₋₆alkynyl.

Other compounds suitable for use in methods of the invention include those of Formula 29, 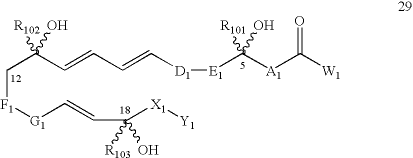
and pharmaceutically acceptable salts, hydrates and solvates thereof, wherein:
D₁-E₁ and F₁-G₁ are independently are cis or trans —C═C— or —C≡C—;
R₁₀₁, R₁₀₂ and R₁₀₃ are independently selected from hydrogen, (C1-C4) straight-chained or branched alkyl, (C2-C4) alkenyl, (C2-C4) alkynyl, (C1-C4) alkoxy, —CH₂R₁₀₄, —CHR₁₀₄R₁₀₄ and —CR₁₀₄R₁₀₄R₁₀₄;
each R₁₀₄ is independently selected from CN, —NO₂ and halogen;
W₁ is selected from —R₁₀₅, —OR₁₀₅, —SR₁₀₅ and —NR₁₀₅R₁₀₅;
each R₁₀₅ is independently selected from hydrogen, (C1-C6) alkyl, (C2-C6) alkenyl or (C2-C6) alkynyl optionally substituted with one or more of the same or different R groups, (C5-C14) aryl optionally substituted with one or more of the same or different R groups, phenyl optionally substituted with one or more of the same or different R groups, (C6-C16) arylalkyl optionally substituted with one or more of the same or different R groups, 5-14 membered heteroaryl optionally substituted with one or more of the same or different R groups, 6-16 membered heteroarylalkyl optionally substituted with one or more of the same or different R groups and a detectable label molecule;
A₁ is selected from (C1-C6) alkylene optionally substituted with 1, 2, 3, 4, 5 or 6 of the same or different halogen atoms, —(CH₂)ₘ—O—CH₂— and —(CH₂)ₘ—S—CH₂—, where m is an integer from 0 to 4;
X₁ is selected from —(CH₂)ₙ— and —(CH₂)ₙ—O—, where n is an integer from 0 to 6;
Y₁ is selected from hydrogen, (C1-C6) alkyl, (C2-C6) alkenyl, or (C2-C6) alkynyl, optionally substituted with one or more of the same or different R₁₀₀ groups, (C5-C14) aryl optionally substituted with one or more of the same or different R₁₀₀ groups, phenyl, optionally substituted with one or more of the same or different R₁₀₀ groups, (C6-C16) arylalkyl optionally substituted with one or more of the same or different R₁₀₀ groups, 5-14 membered heteroaryl optionally substituted with one or more of the same or different R₁₀₀ groups, 6-16 membered heteroarylalkyl optionally substituted with one or more of the same or different R₁₀₀ groups and a detectable label molecule;
each R₁₀₀ is independently selected from an electronegative group, ═O, —OR^{a1}, (C1-C3) haloalkyloxy, ═S, —SR^{a1}, ═NR^{a1}, ═NONR^{a1}, —NR^{c1}R^{c1}, halogen, —CF₃, —CN, —NC, —OCN, —SCN, —NO, —NO₂, ═N₂, —N₃, —S(O)R^{a1}, —S(O)₂R^{a1}, —S(O)₂OR^{a1}, —S(O)₂NR^{c1}R^{c1}, —OS(O)R^{a1}, —OS(O)₂R^{a1}, —OS(O)₂OR^{a1}, —OS(O)₂NR^{c1}R^{c1}, —C(O)R^{a1}, —C(O)OR^{a1}, —C(O)NR^{c1}R^{c1}, —C(NH)NR^{c1}R^{c1}, —OC(O)R^{a1}, —OC(O)OR^{a1}, —OC(O)NR^{c1}R^{c1}, —OC(NH)NR^{c1}R^{c1}, —NHC(O)R^{a1}, —NHC(O)OR^{a1}, —NHC(O)NR^{c1}R^{c1} and —NHC(NH)NR^{c1}R^{c1};
each R^{a1} is independently selected from hydrogen, (C1-C4) alkyl, (C2-C4) alkenyl or (C2-C4) alkynyl; and
each R^{c1} is independently an R^{a1} or, alternatively, R^{c1}R^{c1} taken together with the nitrogen atom to which it is bonded forms a 5 or 6 membered ring.

In certain embodiments of Formula 29, when X₁—Y₁ is —CH₂CH₃, then at least one of R₁₀₁, R₁₀₂ or R₁₀₃ is other than hydrogen.

In certain embodiments, a compound of Formula 29 is represented by Formula 30,

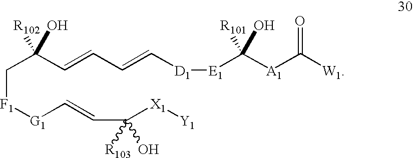

Other compounds suitable for use in methods of the invention include those of Formulae 31 to 37

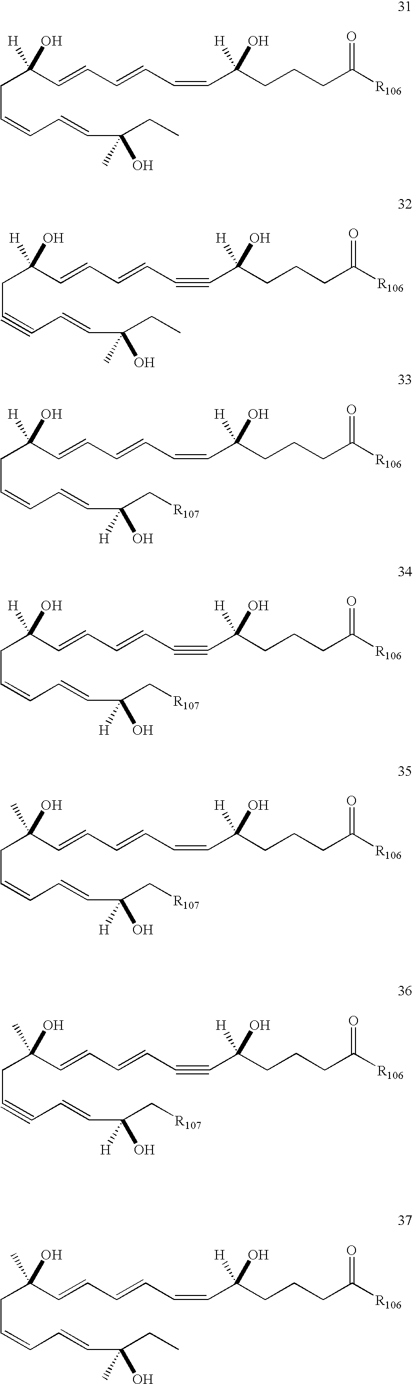

and pharmaceutically acceptable salts, hydrates and solvates thereof,

wherein

R₁₀₆ is —OH, —OCH₃, —OCH(CH₃)₂ or —NHCH₂CH₃; and
R₁₀₇ is 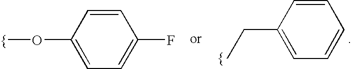
   .

Other compounds suitable for use in methods of the invention include those of Formula 38, 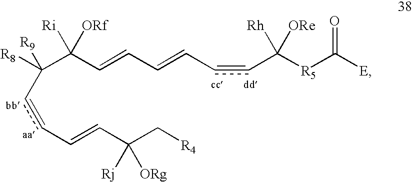
wherein
Carbons aa′ and bb′ are connected by a double bond or a triple bond;
Carbons cc′ and dd′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
E is hydroxyl, alkoxy, aryloxy, amino, alkylamino, dialkylamino, or arylamino;
Rh, Ri and Rj are independently selected from hydrogen, alkyl, alkenyl, alkynyl, perfluoroalkyl, aryl or heteroaryl;
R₄ is selected from hydrogen, alkyl, perfluoroalkyl, alkenyl, alkynyl, aryl, heteroaryl, fluoro, hydroxyl, alkoxy, aryloxy;
R₅ is selected from i-iv as follows: i) CH₂CH(R₆)CH₂, where R₆ is hydrogen, alkyl, alkenyl, alkynyl, perfluoroalkyl, aryl, heteroaryl, fluoro, hydroxyl or alkoxy; ii) CH₂C(R₆R₇)CH₂, where R₆ and R₇ are each independently alkyl, alkenyl, alkynyl, perfluoroalkyl, aryl, or fluoro, or R₆ and R₇ are connected together to form a carbocyclic or heterocyclic ring; iii) CH₂OCH₂, CH₂C(O)CH₂, or CH₂CH₂; or iv) R₅ is a carbocyclic, heterocyclic, aryl or heteroaryl ring; and
R₈ and R₉ are independently selected from hydrogen, alkyl, alkenyl, alkynyl, perfluoroalkyl, alkoxy, aryl or heteroaryl, or R₈ and R₉ are connected together to form a carbocyclic or heterocyclic ring;
   or pharmaceutically acceptable salts thereof.

In certain embodiments R₈ and R₉ are hydrogen.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Other compounds suitable for use in methods of the invention include those of Formulas 39-44, 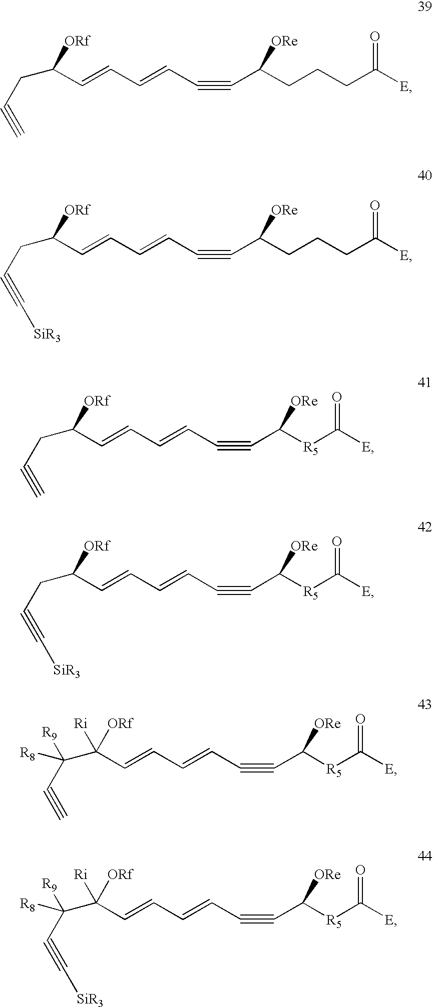
and pharmaceutically acceptable salts thereof, wherein
R^{e}, R^{f}, E, Ri, R₅, R₈ and R₉ are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

The compounds above (e.g., compounds of formula A or formulae 1 to 44) are known to be useful in the treatment or prevention of inflammation or inflammatory disease. Examples of such compounds are disclosed in the following patents and applications: US 2003/0191184, WO 2004/014835, WO 2004/078143, U.S. Pat. No. 6,670,396, US 2003/0236423, US 2005/0228047, and US2005/0261255. These compounds are suitable for use in methods of the present invention.

Other compounds useful in this invention are compounds that are chemically similar variants to any of the compounds of formula A or formulae 1 to 44 set forth above. The term “chemically similar variants” includes, but is not limited to, replacement of various moieties with known biosteres; replacement of the end groups of one of the compounds above with a corresponding end group of any other compound above, modification of the orientation of any double bond in a compound, the replacement of any double bond with a triple bond in any compound, and the replacement of one or more substituents present in one of the compounds above with a corresponding substituent of any other compound.

Lipoxin compounds suitable for use in this invention include those of formula 50:

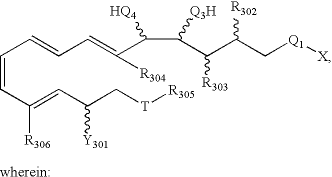

X is R₃₀₁, OR₃₀₁, or SR₃₀₁;

R₃₀₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl 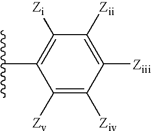
   wherein Zᵢ, Zᵢᵢ, Zᵢᵢᵢ, Zᵢᵥ and Zᵥ are each independently selected from —NO₂, —CN, —C(═O)—R₃₀₁, —SO₃H, a hydrogen atom, halogen, methyl, —ORₓ, wherein Rₓ is 1 to 8 carbon atoms, inclusive, which may be a straight chain or branched, and hydroxyl, wherein when any of Zᵢ Zᵢᵢ, Zᵢᵢᵢ, Zᵢᵥ or Zᵥ is C(═O)—R₃₀₁, said Zᵢ Zᵢᵢ, Zᵢᵢᵢ, Zᵢᵥ or Zᵥ is not substituted with another C(═O)—R₃₀₁.
(g) a detectable label molecule; or
(h) a straight or branched chain alkenyl of 2 to 8 carbon atoms, inclusive;

Q₁ is (C═O), SO₂ or (CN), provided when Q₁ is CN, then X is absent;

Q₃ and Q₄ are each independently O, S or NH;

one of R₃₀₂ and R₃₀₃ is a hydrogen atom and the other is:
(a) H;
(b) an alkyl of 1 to 8 carbon atoms, inclusive, which may be a straight chain or branched;
(c) a cycloalkyl of 3 to 6 carbon atoms, inclusive;
(d) an alkenyl of 2 to 8 carbon atoms, inclusive, which may be straight chain or branched; or
(e) RₖQ₂Rₗ wherein Q₂ is —O— or —S—; wherein Rₖ is alkylene of 0 to 6 carbons atoms, inclusive, which may be straight chain or branched and wherein Rₗ is alkyl of 0 to 8 carbon atoms, inclusive, which may be straight chain or branched, provided when Rₗ is 0, then Rₗ is a hydrogen atom;

R₃₀₄ is
(a) H;
(b) an alkyl of 1 to 6 carbon atoms, inclusive, which may be a straight chain or branched;

R₃₀₅ is 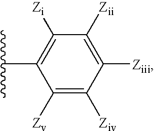
wherein Zᵢ Zᵢᵢ, Zᵢᵢᵢ, Zᵢᵥ and Zᵥ are defined as above;

R₃₀₆ is
(a) H;
(b) an alkyl from 1 to 4 carbon atoms, inclusive, straight chain or branched;

wherein Y₃₀₁ is —OH, methyl, —SH, an alkyl of 2 to 4 carbon atoms, inclusive, straight chain or branched, an alkoxy of 1 to 4 carbon atoms, inclusive, or (CH)ₚ(Z)_{q}, where p+q=3, p=0 to 3, q=0 to 3 and Z is cyano, nitro or a halogen; and

T is O or S;

and pharmaceutically acceptable salts thereof.

Lipoxin compounds suitable for use in this invention include those of formulae 51, 52, 53 or 54:

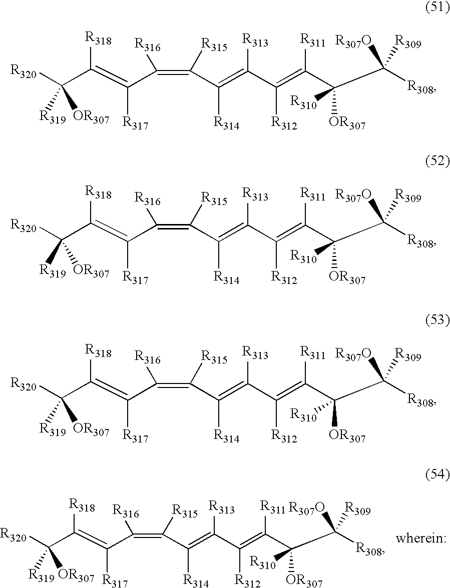

each R₃₀₇ is independently selected from hydrogen and straight, branched, cyclic, saturated, or unsaturated alkyl having from 1 to 20 carbon atoms;

R₃₀₈, R₃₀₉, R₃₁₀, R₃₁₉, and R₃₂₀ are independently selected from:
(a) hydrogen;
(b) straight, branched, cyclic, saturated, or unsaturated alkyl having from 1 to 20 carbon atoms;
(c) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(d) substituted aryl or heteroaryl, wherein the aryl or heteroaryl is substituted with one or more substituents selected from alkyl, cycloalkyl, alkoxy, halo, aryl, heteroaryl, carboxyl, and carboxamido; and
(e) Z-Y, wherein:

Z is selected from a straight, branched, cyclic, saturated, or unsaturated alkyl having from 1 to 20 carbon atoms; substituted lower alkyl, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; and substituted aryl or heteroaryl, wherein the aryl or heteroaryl is substituted with one or more substituents selected from alkyl, cycloalkyl, alkoxy, halo, aryl, heteroaryl, carboxyl, and carboxamido; and

Y is selected from hydrogen; alkyl; cycloalkyl; carboxyl; carboxamido; aryl; heteroaryl; substituted aryl or heteroaryl, wherein the aryl or heteroaryl is substituted with one or more substituents selected from alkyl, cycloalkyl, alkoxy, halo, aryl, heteroaryl, carboxyl, and carboxamido; and

R₃₁₁ to R₃₁₈ are independently selected from:
(a) hydrogen;
(b) halo;
(c) straight, branched, cyclic, saturated, or unsaturated alkyl having from 1 to 20 carbon atoms;
(d) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(e) substituted aryl or heteroaryl, wherein the aryl or heteroaryl is substituted with one or more substituents selected from alkyl, cycloalkyl, alkoxy, halo, aryl, heteroaryl, carboxyl, and carboxamido; or

R₃₀₈ to R₃₂₀ are independently a bond that forms a carbon-carbon double bond, a carbon-carbon triple bond, or a ring with the lipoxin backbone; or

any two of R₃₀₇ to R₃₂₀ are taken together with the atoms to which they are bound and optionally to 1 to 6 oxygen atoms, 1 to 6 nitrogen atoms, or both 1 to 6 oxygen atoms and 1 to 6 nitrogen atoms, to form a ring containing 3 to 20 atoms.

Lipoxin compounds suitable for use in this invention include those of formula 55:

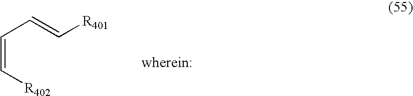

R₄₀₁ is selected from:

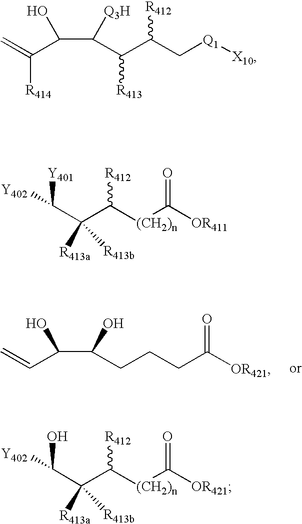

R₄₀₂ is selected from:

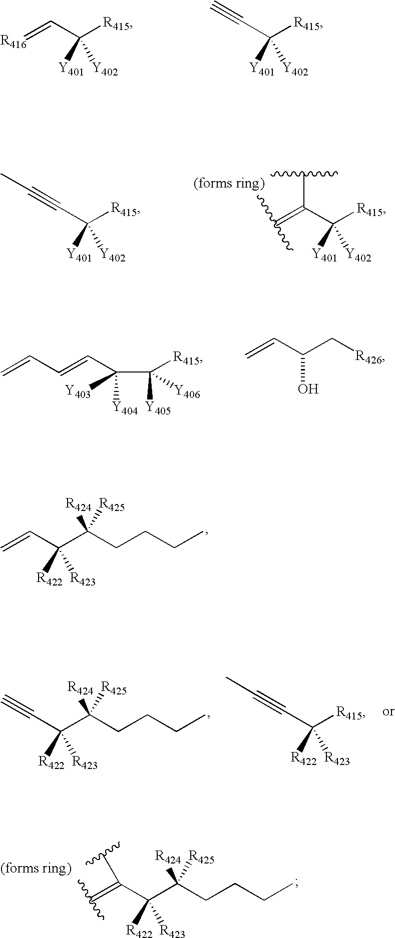

X₁₀ is R₄₁₁, OR₄₁₁, or SR₄₁₁;

R₄₁₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl 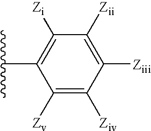
   wherein Zᵢ Zᵢᵢ, Zᵢᵢᵢ, Zᵢᵥ and Zᵥ are each independently selected from —NO₂, —CN, —C(═O)—R₄₁₁, —SO₃H, a hydrogen atom, halogen, methyl, —ORₓ, wherein Rₓ is 1 to 8 carbon atoms, inclusive, which may be a straight chain or branched, and hydroxyl; wherein when any of Zᵢ Zᵢᵢ, Zᵢᵢᵢ, Zᵢᵥ or Zᵥ is C(═O)—R₄₁₁, said Zᵢ Zᵢᵢ, Zᵢᵢᵢ, Zᵢᵥ or Zᵥ is not substituted with another C(═O)—R₄₁₁.
(g) a detectable label molecule; or
(h) a straight or branched chain alkenyl of 2 to 8 carbon atoms, inclusive;

Q₁ is (C═O), SO₂ or (CN);

Q₃ is O, S or NH;

one of R₄₁₂ and R₄₁₃ is a hydrogen atom and the other is selected from:

(a) H;
(b) an alkyl of 1 to 8 carbon atoms, inclusive, which can be straight chain or branched;
(c) a cycloalkyl of 3 to 6 carbon atoms, inclusive;
(d) an alkenyl of 2 to 8 carbon atoms, inclusive, which can be straight chain or branched; or
(e) R₄₃₁Q₂R₄₃₂ wherein Q₂ is —O— or —S—; wherein R₄₃₁ is alkylene of 0 to 6 carbons atoms, inclusive, which can be straight chain or branched and wherein R₄₃₁ is alkyl of 0 to 8 carbon atoms, inclusive, which can be straight chain or branched;

R₄₁₃ₐ and R_{413b} are each independently:
(a) H;
(b) an alkyl of 1 to 8 carbon atoms, inclusive, which can be straight chain or branched;
(c) a cycloalkyl of 3 to 6 carbon atoms, inclusive;
(d) an alkenyl of 2 to 8 carbon atoms, inclusive, which can be straight chain or branched; or
(e) R₄₃₁Q₂R₄₃₂ wherein R₄₃₁, Q₂, and R₄₃₂ are as defined above;

R₄₁₄ is
(a) H;
(b) an alkyl of 1 to 6 carbon atoms, inclusive, can be straight chain or branched;

R₄₁₅ is
(a) an alkyl of 1 to 9 carbon atoms which can be straight chain or branched;
(b) —(CH₂)—Rᵢ

wherein n=0 to 4 and Rᵢ is
(i) a cycloalkyl of 3 to 10 carbon atoms, inclusive;
(ii) a phenyl; or
(iii) substituted phenyl 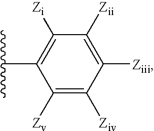
   wherein Zᵢ through Zᵥ are as defined above;
(b) R₄₃₁Q₂R₄₃₂, wherein R₄₃₁, Q₂, and R₄₃₂ are as defined above;
(c) —C(Rᵢᵢᵢ)(Rᵢᵥ)—Rᵢ,

wherein Rᵢᵢᵢ and Rᵢᵥ are each independently:
(i) a hydrogen atom;
(ii) (CH)ₚ(Z)_{q}, wherein Z, p, and q are as defined above;
(e) a haloalkyl of 1 to 8 carbon atoms, inclusive, and 1 to 6 halogen atoms, inclusive, straight chain or branched;

R₄₁₆ is
(a) H;
(b) an alkyl from 1 to 4 carbon atoms, inclusive, straight chain or branched;
(c) a halogen;

one of Y₄₀₁ or Y₄₀₂ is —OH, methyl, or —SH, and wherein the other is selected from:
(a) H;
(b) (CH)ₚ(Z)_{q} where p+q=3, p=0 to 3, q=0 to 3 and each Z, independently, is cyano, nitro or a halogen;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(d)═NH; or
(e)═O;

one of Y₄₀₃ or Y₄₀₄ is —OH, methyl, or —SH, and wherein the other is selected from:
(a) H;
(b) (CH)ₚ(Z)_{q} wherein Z, p, and q are as defined above;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(a) ═NH; or
(b) ═O;

one of Y₄₀₅ or Y₄₀₆ is —OH, methyl, or —SH, and wherein the other is selected from:
(a) H
(b) (CH)ₚ(Z)_{q} wherein Z, p, and q are as defined above;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(a) ═NH; or
(b) ═O;

R₄₂₁ is
(a) H; or
(b) alkyl of 1 to 8 carbon atoms;

R₄₂₂ and R₄₂₃ are each independently:
(a) H;
(b) a hydroxyl, or a thiol;
(c) a methyl or a halomethyl;
(d) a halogen; or
(e) an alkoxy of 1 to 3 carbon atoms;

R₄₂₄ and R₄₂₅ are each independently:
(a) H;
(b) a hydroxyl, or a thiol;
(c) a methyl or a halomethyl;
(d) a halogen;
(e) an alkoxy of 1 to 3 carbon atoms; or
(f) an alkyl or haloalkyl of 2 to 4 carbon atoms inclusive, which can be straight chain or branched; and

R₄₂₆ is
(a) a substituted phenyl 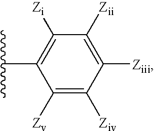
   wherein Zᵢ through Zᵥ are as defined above;
(b) a substituted phenoxy 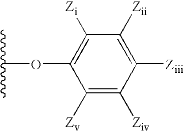
   wherein Zᵢ through Zᵥ are as defined above; or
(c) 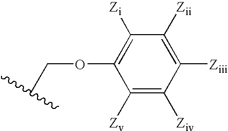
   wherein Zᵢ through Zᵥ are as defined above.

Lipoxin compounds suitable for use in this invention include those of formula 56:

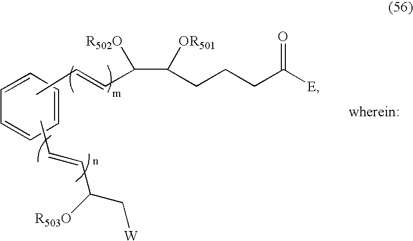

E is hydroxy, alkoxy, aryloxy, amino, alkylamino, dialkylamino or —OM, where M is a cation selected from ammonium, tetra-alkyl ammonium, and the cations of sodium, potassium, magnesium and zinc;

W is hydrogen, alkyl, alkenyl, alkynyl, aryl, heteroaryl, halo, hydroxy, alkoxy, aryloxy, carboxy, amino, alkylamino, dialkylamino, acylamino, carboxamido, or sulfonamide;

each of R₅₀₁-R₅₀₃ are independently selected from hydrogen, alkyl, aryl, acyl or alkoxyacyl;

n is 0, 1 or 2;

m is 1 or 2; and

the two substituents on the phenyl ring are ortho, meta, or para.

Lipoxin compounds suitable for use in this invention include those of formula 57:

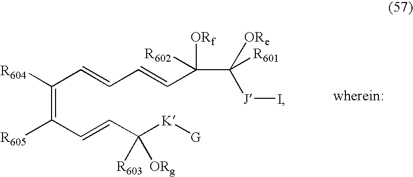

I is selected from: —C(O)-E, —SO₂-E, —PO(OR)-E, where E is hydroxy, alcoxy, aryloxy, amino, alkylamino, dialkylamino, or —OM, where M is a cation selected from ammonium, tetra-alkyl ammonium, Na, K, Mg, and Zn; and R is hydroxyl or alkoxy

J′ and K′ are linkers independently selected from a chain of up to 20 atoms and a ring containing up to 20 atoms, provided that J′ and K′ can independently include one or more nitrogen, oxygen, sulfur or phosphorous atoms, and further provided that J′ and K′ can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J′ and K′ can also contain one or more fused carbocyclic, heterocyclic, aryl or heteroaryl rings, and provided that linkers J′ and K′ are connected to the adjacent C(R)OR group via a carbon atom or a C-heteroatom bond where the heteroatom is oxygen, sulfur, phosphorous or nitrogen;

G is selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.

Re, Rf and Rg, are independently selected from hydrogen, alkyl, aryl, heteroaryl, acyl, silyl, alcoxyacyl and aminoacyl;

R₆₀₁, R₆₀₂ and R₆₀₃ are independently selected from hydrogen, alkyl, aryl and heteroaryl, provided that R₆₀₁, R₆₀₂ and R₆₀₃ can independently be connected to linkers J′ or K′;

R₆₀₄ and R₆₀₅ are independently selected from hydrogen, alkyl, alkenyl, alkynyl, aryl, heteroaryl, fluoro, and provided that R₆₀₄ and R₆₀₅ can be joined together to form a carbocyclic, heterocyclic or aromatic ring, and further provided that R₆₀₄ and R₆₀₅ can be replaced by a bond to form a triple bond.

Other compounds suitable for use in methods of the invention are the oxylipins described in international application WO 2006055965, the compounds in which are incorporated herein by reference. Examples of such compounds are those of formulae 58 to 115, as shown in Table 1. These compounds include long chain omega-6 fatty acids, docosapentaenoic acid (DPAn-6) (compounds 58-73) and docosatetraenoic acid (DTAn-6) (compounds 74-83), and the omega-3 counterpart of DPAn-6, docosapentaenoic acid (DPAn-3) (compounds 84-97). Further compounds are the docosanoids 98-115.

TABLE 1

| | |
|---|---|
| 10,17- Dihydroxy DPAn-6 (58) | 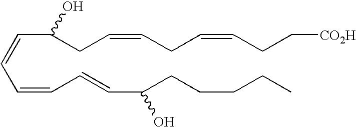 |
| 16,17- Dihydroxy DPAn-6 (59) | 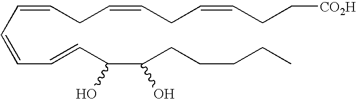 |
| 4,5- Dihydroxy DPAn-6 (60) | 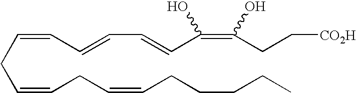 |
| 7,17- Dihydroxy DPAn-6 (61) | 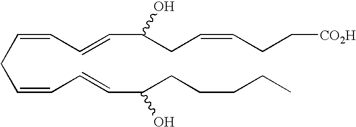 |
| 7-Hydroxy DPAn-6 (62) | 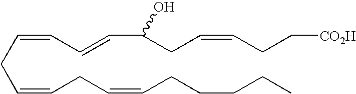 |
| 10- hydroxy DPAn-6 (63) | 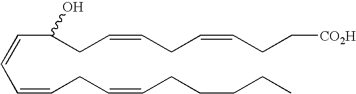 |
| 13- Hydroxy DPAn-6 (64) | 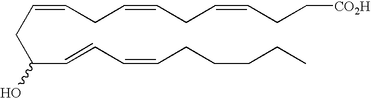 |
| 17- hydroxy DPAn-6 (65) | 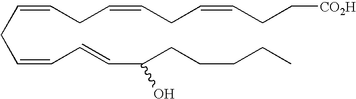 |
| 4,5,17- Tri- hydroxy DPAn-6 (66) | 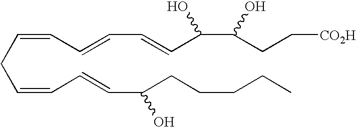 |
| 7,16,17- Tri- hydroxy DPAn-6 (67) | 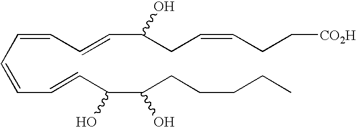 |
| 8-Hydroxy DPAn-6 (68) | 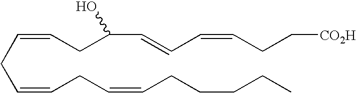 |
| 14- Hydroxy DPAn-6 (69) | 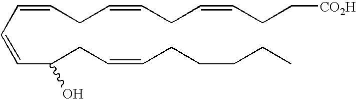 |
| 13,17- Dihydroxy DPAn-6 (70) | 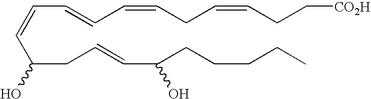 |
| 7,14- Dihydroxy DPAn-6 (71) | 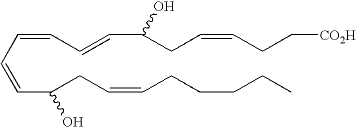 |
| 8,14- Dihydroxy DPAn-6 (72) | 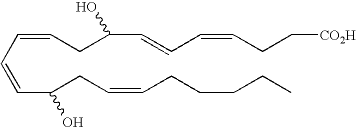 |
| 11- Hydroxy DPAn-6 (73) | 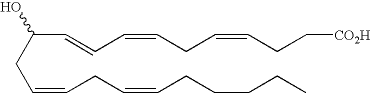 |
| 10,17- Di- hydroxy- DTAn-6 (74) | 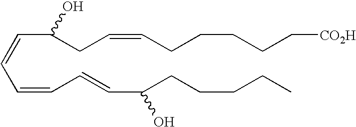 |
| 16,17- Di- hydroxy- DTAn-6 (75) | 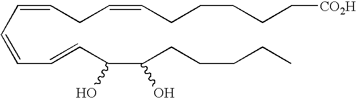 |
| 4,5- Di- hydroxy- DTAn-6 (76) | 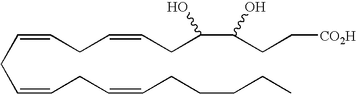 |
| 7,17- Di- hydroxy- DTAn-6 (77) | 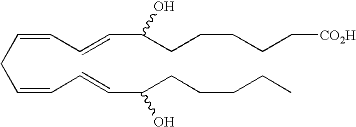 |
| 7- Hydroxy- DTAn-6 (78) | 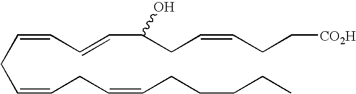 |
| 10- Hydroxy- DTAn-6 (79) | 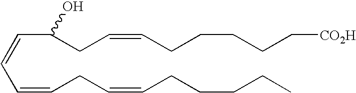 |
| 13- Hydroxy- DTAn-6 (80) | 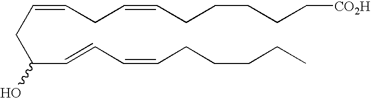 |
| 17- Hydroxy- DTAn-6 (81) | 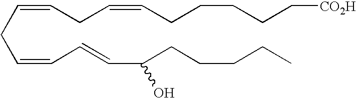 |
| 4,5,17- Tri- hydroxy- DTAn-6 (82) | 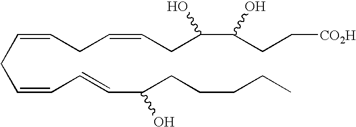 |
| 7,16,17- Tri- hydroxy- DTAn-6 (83) | 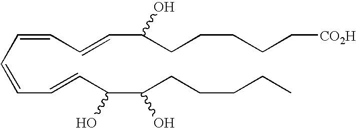 |
| 10,17- Dihydroxy DPAn-3 (84) | 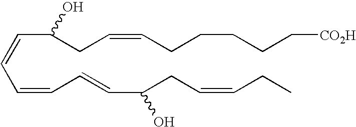 |
| 10,20- Dihydroxy DPAn-3 (85) | 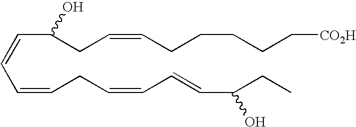 |
| 13,20- Dihydroxy DPAn-3 (86) | 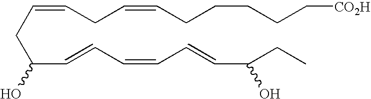 |
| 16,17- Dihydroxy DPAn-3 (87) | 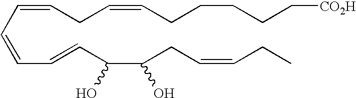 |
| 7,17- Dihydroxy DPAn-3 (88) | 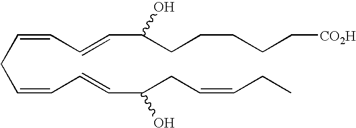 |
| 7-Hydroxy DPAn-3 (89) | 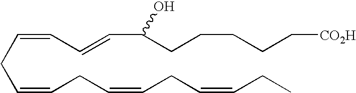 |
| 10- Hydroxy DPAn-3 (90) | 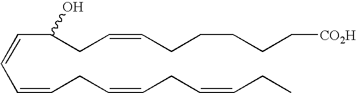 |
| 13- Hydroxy DPAn-3 (91) | 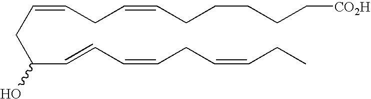 |
| 17- Hydroxy DPAn-3 (92) | 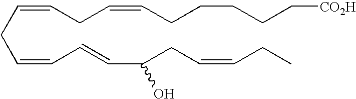 |
| 7,16,17- Tri- hydroxy DPAn-3 (93) | 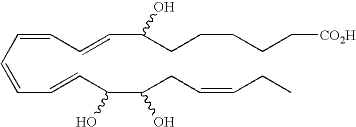 |
| 16- Hydroxy DPAn-3 (94) | 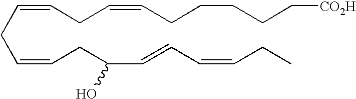 |
| 11- Hydroxy DPAn-3 (95) | 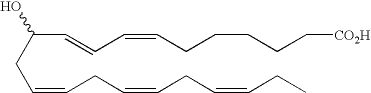 |
| 14- Hydroxy DPAn-3 (96) | 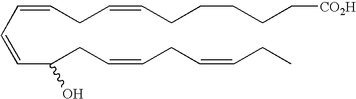 |
| 8,14- Dihydroxy DPAn-3 (97) | 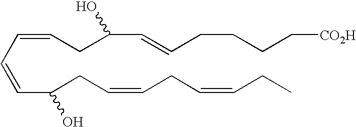 |
| 10,11- Epoxy DHA (98) | 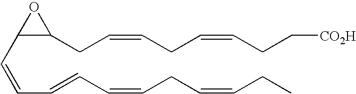 |
| 13,14- Dihydroxy DHA (99) | 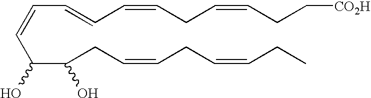 |
| 13,14- Epoxy DHA (100) | 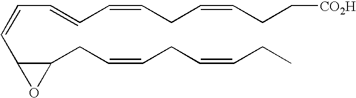 |
| 19,20- Epoxy DHA (101) | 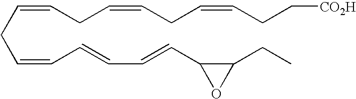 |
| 7,8- Epoxy DHA (102) | 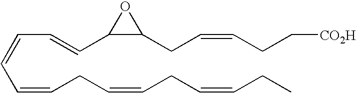 |
| 4,5- Epoxy- 17-OH DPA (103) | 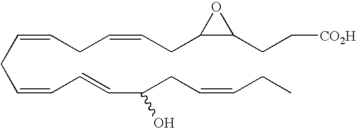 |
| 7,16,17- Tri- hydroxy DTAn-3 (104) | 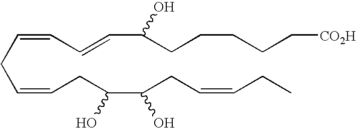 |
| 16,17- Dihydroxy DTAn-3 (105) | 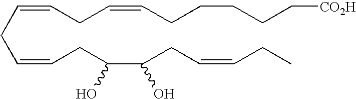 |
| 10,16,17- Tri- hydroxy DTRAn-6 (106) | 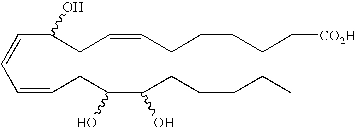 |
| 16,17- Dihydroxy DTRAn-6 (107) | 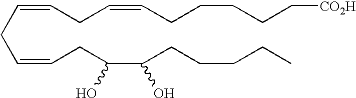 |
| 7,16,17- Tri- hydroxy DTRAn-6 (108) | 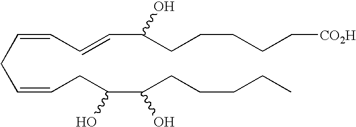 |
| 15-epi- lipoxin A4 (109) | 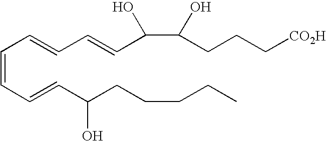 |
| 16,17- epoxy DHA (110) | 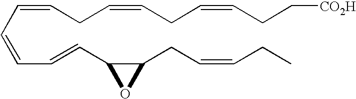 |
| 7,8- epoxy DPA (111) | 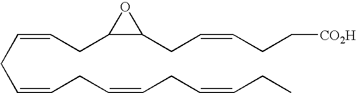 |
| 10,11 epoxy DPA (112) | 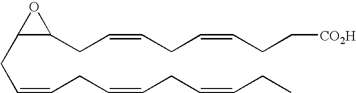 |
| 19,20 epoxy DPA (113) | 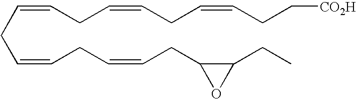 |
| 7-hydroxy DHA (114) | 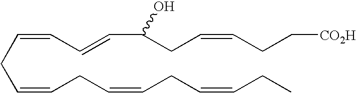 |
| 13,14 epoxy DPA (115) | 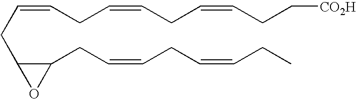 |

Other oxylipin compounds that are suitable for use in methods of the invention include analogs of the compounds shown in Table 1. Such compounds include but are not limited to those analogs wherein one or more double bonds are replaced by triple bonds, those wherein carboxy groups are derivatized to form esters, amides or salts, those wherein the hydroxyl-bearing carbons are further derivatized (with, for example, a substituted or unsubstituted, branched or unbranched alkyl, alkenyl, or alkynyl group, substituted or unsubstituted aryl group, substituted or unsubstituted, branched or unbranched alkylaryl group, halogen atom) to form tertiary alcohols (or ethers, esters, or other derivatives thereof), those wherein one or more hydroxyl groups are derivatized to form esters or protected alcohols, or those having combinations of any of the foregoing modifications.

Further oxylipin compounds suitable for use in methods of the invention include the following: isolated docosanoids of docosapentaenoic acid (DPAn-6); monohydroxy, dihydroxy, and trihydroxy derivatives of DPAn-6; isolated docosanoids of docosapentaenoic acid (DPAn-3); monohydroxy, dihydroxy, and trihydroxy derivatives of DPAn-3; isolated docosanoids of docosapentaenoic acid (DTAn-6); or monohydroxy, dihydroxy, and trihydroxy derivatives of DTAn-6.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. Co alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group 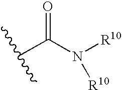
wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by 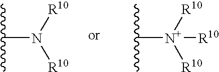
wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group 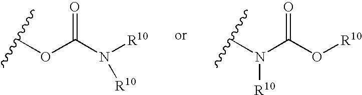
wherein each R¹⁰ independently represent hydrogen or a hydrocarbyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae 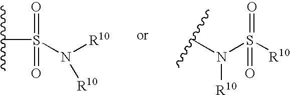
wherein each R¹⁰ independently represents hydrogen or hydrocarbyl.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula 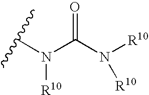
wherein each R¹⁰ independently represent hydrogen or a hydrocarbyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The synthesis of each of the statins and each of the compounds of formula A, compounds of any one of formulae 1 to 44, lipoxin compounds, or oxylipin compounds set forth above can be achieved by methods well-known in the art. For example, the synthesis of compounds of formula A or formulae 1 to 44 is set forth in US 2003/0191184, WO 2004/014835, WO 2004/078143, U.S. Pat. No. 6,670,396, US 2003/0236423 and US 2005/0228047, all of which are herein incorporated by reference. The synthesis of various statins is set forth in U.S. Pat. No. RE37314 E, U.S. Pat. No. 4,444,784, U.S. Pat. No. 4,346,227, U.S. Pat. No. 5,354,772, U.S. Pat. No. 4,681,893, US 2005/0261255 and US 2005/0228042. The synthesis of lipoxin compounds is set forth in US 2002/0107289, US 2004/0019110, US 2006/0009521, US 2005/0203184, US 2005/0113443. The preparation of oxylipin compounds is set forth in WO 2006/055965.

In another embodiment, the invention provides a method of raising serum HDL concentration (or preventing a decrease in serum HDL concentration) or decreasing the serum LDL/HDL ratio in a patient, said method comprising administering to said patient a compound of formula A, a compound of any one of formulae 1 to 44, a lipoxin compound, or an oxylipin compound, or a combination of aspirin and an omega-3 fatty acid. The patient to be treated in this method may have a total serum cholesterol level of greater than 189 mg/dl, preferably higher than 200 mg/dl and most preferably higher than 240 mg/dl; and/or a serum LDL concentration of greater than 130 mg/dl, preferably greater than 160 mg/dl, and most preferably higher than 189 mg/dl. In addition to serum cholesterol and/or LDL levels, other factors to be considered are the presence or absence of coronary disease and risk factors, such as age (45 or over for men, 55 or over for women), family history of coronary heart disease, smoking, high blood pressure, serum HDL cholesterol level, or presence of diabetes.

In certain embodiments, the patient to be treated in this method of the invention may already be receiving a cholesterol-lowering drug. In one preferred embodiment, the patient is already taking a statin, such as one of the statins described above; and will continue to take that drug conjointly with a compound of formula A, a compound of any one of formulae 1 to 44, a lipoxin compound, or an oxylipin compound, or a combination of aspirin and an omega-3 fatty acid. Alternatively, the compound of formula A, compound of any one of formulae 1 to 44, lipoxin compound, or oxylipin compound, or the combination of aspirin and an omega-3 fatty acid may be used as a replacement for the previously administered cholesterol-lowering drug.

In a related embodiment, the invention provides a method of reducing the dose of a statin required to achieve a desired increase in serum HDL, or a decrease in serum LDL/HDL ratio or serum total cholesterol level. Reducing the dose of statins while maintaining potent serum lipid-reducing properties is highly desirable due to side effects associated with certain statins. Well-known side effects include, deleterious changes in liver function, muscle pain, weakness, muscle tenderness, myopathy. Other side effects of statins include reduced cognition, memory impairment, depression, irritability, non-muscle pain, peripheral neuropathy, sleep disorders, sexual dysfunction, fatigue, dizziness, swelling, shortness of breath, vision changes, changes in temperature regulation, weight change, hunger, breast enlargement, blood sugar changes, dry skin, rashes, blood pressure changes, nausea, upset stomach, bleeding, and ringing in ears or other noises.

In this embodiment, the dose of a statin is reduced by at least 5%, at least 10%, at least 15%, at least 20%, at least 25%, at least 30%, at least 40%, at least 50%, at least 60%, at least 70%, or more. The actual reduction in statin dose will depend upon the nature of the compound of formula A, compound of any one of formulae 1 to 44, lipoxin compound, oxylipin compound, or combination of aspirin and an omega-3 fatty acid being administered, the amount of compound of formula A, compound of any one of formulae 1 to 44, lipoxin compound, oxylipin compound, or combination of aspirin and an omega-3 fatty acid being administered, and the reduction in serum lipid level desired, as well as other factors set forth elsewhere in this application that are typically considered in treating a disease or condition. The amount of compound of formula A, compound of any one of formulae 1 to 44, lipoxin compound, or oxylipin compound, or combination of aspirin and an omega-3 fatty acid administered in this method will also depend upon the factors set forth above, as well as the nature and amount of statin being administered. In certain embodiments, the amount of compound of formula A, compound of any one of formulae 1 to 44, lipoxin compound, or oxylipin compound, or combination of aspirin and an omega-3 fatty acid administered in this method is less than 5%, less than 10%, less than 15%, less than 20%, less than 25%, less than 30%, less than 40%, less than 50%, less than 60%, less than 70%, less than 80%, or less than 90% of the dose of compound of formula A, compound of any one of formulae 1 to 44, lipoxin compound, oxylipin compound, or combination of aspirin and an omega-3 fatty acid required to produce an anti-inflammatory effect. In other embodiments, the amount of compound of formula A, compound of any one of formulae 1 to 44, lipoxin compound, or oxylipin compound, or combination of aspirin and an omega-3 fatty acid administered is over 110%, over 120%, over 130%, over 140%, over 150%, over 160%, over 170%, over 180%, over 190%, or even over 200% of the dose of compound of formula A, compound of any one of formulae 1 to 44, lipoxin compound, oxylipin compound, or combination of aspirin and an omega-3 fatty acid required to produce an anti-inflammatory effect.

In yet another embodiment, the invention provides a composition comprising a statin, a compound of formula A, a compound of any one of formulae 1 to 44, a lipoxin compound, or an oxylipin compound, or a combination of aspirin and an omega-3 fatty acid, and a pharmaceutically acceptable carrier. In these compositions, the statin may be selected from any statin known in the art, preferably one of the statins set forth above. Similarly, the compound of formula A or of any of formulae 1 to 44 may be selected from any such compound known in the art, such one of the compounds set forth above. Similarly, the lipoxin may be selected from any lipoxin known in the art, preferably one of the lipoxins set forth above. Similarly, the oxylipin may be selected from any oxylipin known in the art, preferably one of the oxylipins set forth above. The amount of statin in this combination composition is less than 5%, less than 10%, less than 15%, less than 20%, less than 25%, less than 30%, less than 40%, less than 50%, less than 60%, less than 70%, less than 80%, less than 90%, or less than 100% of the amount of statin normally administered in a single dosage (monotherapy) to reduce serum lipid concentration. Preferably, the amount of statin is less than 90%, more preferably less than 80%, and most preferably, less than 70% of the recommended monotherapy dosage amount. The amount of compound of formula A, compound of any one of formulae 1 to 44, lipoxin compound, or oxylipin compound, or combination of aspirin and an omega-3 fatty acid in the combination composition of this invention is less than 5%, less than 10%, less than 15%, less than 20%, less than 25%, less than 30%, less than 40%, less than 50%, less than 60%, less than 70%, less than 80%, less than 90%, or less than 100% of the dose of compound of formula A, compound of any one of formulae 1 to 44, lipoxin compound, oxylipin compound, or combination of aspirin and an omega-3 fatty acid administered in a single dosage to produce an anti-inflammatory effect. In other embodiments, the amount of compound of formula A, compound of any one of formulae 1 to 44, lipoxin compound, or oxylipin compound, or combination of aspirin and an omega-3 fatty acid in the combination composition of this invention is over 100%, over 110%, over 120%, over 130%, over 140%, over 150%, over 160%, over 170%, over 180%, over 190%, or even over 200% of the dose of compound of formula A, compound of any one of formulae 1 to 44, lipoxin compound, oxylipin compound, or combination of aspirin and an omega-3 fatty acid required to produce an anti-inflammatory effect. Preferably, the amount of compound of formula A, compound of any one of formulae 1 to 44, lipoxin compound, oxylipin compound, or combination of aspirin and an omega-3 fatty acid is less than 100%, preferably less than 90%, more preferably less than 80% and most preferably, less than 70% of the dose of compound of formula A, compound of any one of formulae 1 to 44, lipoxin compound, oxylipin compound, or combination of aspirin and an omega-3 fatty acid administered in a single dosage to produce an anti-inflammatory effect.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of formula A, compound of any one of formulae 1 to 44, lipoxin compound, oxylipin compound, or aspirin and/or an omega-3 fatty acid and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

A pharmaceutically acceptable carrier can contain physiologically acceptable agents that act, for example, to stabilize or to increase the absorption of a compound such as a compound of formula A, compound of any one of formulae 1 to 44, lipoxin compound, oxylipin compound, or aspirin and/or an omega-3 fatty acid. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramusclularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of formula A, a compound of any one of formulae 1 to 44, a lipoxin compound, an oxylipin compound, or aspirin and/or an omega-3 fatty acid, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, the suitable daily dose of a compound of formula A, a compound of any one of formulae 1 to 44, a lipoxin compound, an oxylipin compound, or a combination of aspirin and an omega-3 fatty acid for treating non-inflammatory cardiovascular disease will be 2 times, 5 times, 10 times, or 20 times more than the dose administered for treating inflammation. In certain embodiments, the suitable daily dose of a compound of formula A, a compound of any one of formulae 1 to 44, a lipoxin compound, an oxylipin compound, or a combination of aspirin and an omega-3 fatty acid for treating non-inflammatory cardiovascular disease will be 2 times, 5 times, 10 times, or 20 times less than the dose administered for treating inflammation.

In certain embodiments, the method of treating cardiovascular disease may comprise administering a compound of formula A, a compound of any one of formulae 1 to 44, a lipoxin compound, or an oxylipin compound, or a combination of aspirin and an omega-3 fatty acid alone, conjointly with a statin and/or conjointly with another therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In one embodiment, the method of treating cardiovascular disease according to this invention may comprise the additional step of conjointly administering to the patient another cardiovascular agent including, for example, a cycloogenase inhibitor, a thromboxane receptor antagonist, a prostacyclin mimetic, a phosphodiesterase inhibitor, a vasodilator, a cerebral protecting drug, a brain metabolic stimulant, an anticoagulant, an antiplatelet drug, a thrombolytic drug, an antihypertensive agent, a calcium channel blocker, an antianginal drug, a diuretic, a cardioplegic solution, a cardiotonic agent, an antiarrhythmic drug, a fibrinolytic agent, a sclerosing solution, a vasoconstrictor agent, a nitric oxide donor, a potassium channel blocker, a sodium channel blocker, an antihyperlipidemic drug, an immunosuppressant, or a naturiuretic agent.

Examples of a cyclooxygenase inhibitor include aspirin or indomethacin.

An example of a thromboxane receptor antagonist is ifetroban.

Examples of vasodilators include, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin.

Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs.

Examples of the brain metabolic stimulants include amantadine, tiapride, and gamma-aminobutyric acid.

Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate.

Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene.

Examples of the thrombolytic drug include urokinase, tissue plasminogen activator (tPA), recombinant tPA, issue-type plasminogen activators (such as alteplase, tisokinase, nateplase, pamiteplase, monteplase, and rateplase), streptokinase, urokinase, prourokinase, anisoylated plasminogen streptokinase activator complex (APSAC, Eminase, Beecham Laboratories), animal salivary gland plasminogen activators, and nasaruplase.

Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, zofenopril, pentopril, randolapril and salts of such compounds), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of the antianginal drug include nitrate drugs (such as amyl nitrite, nitroglycerin, and isosorbide), β-adrenaline receptor blocking drugs (exemplified above), calcium channel blocking drugs (exemplified above) trimetazidine, dipyridamole, etafenone, dilazep, trapidil, nicorandil, enoxaparin, and aspirin.

Examples of the diuretic include thiazide diuretics (such as hydrochlorothiazide, methyclothiazide, bendrofluazide, chlorothiazide, trichlormethiazide, benzylhydrochlorothiazide, flumethiazide, hydroflumethiazide, bendroflumethiazide, methylchlorthiazide, polythiazide, benzthiazide and penflutizide), loop diuretics (such as furosemide, etacrynic acid, bumetamide, piretamide, azosemide, and torasemide), K⁺ sparing diuretics (spironolactone, triamterene, amiloride, and potassium canrenoate), osmotic diuretics (such as isosorbide, D-mannitol, and glycerin), nonthiazide diuretics (such as meticrane, tripamide, chlorthalidone, and mefruside), and acetazolamide.

Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate.

Examples of the antiarrhythmic drug include ajmaline, pirmenol, procainamide, cibenzoline, disopyramide, quinidine, aprindine, mexiletine, lidocaine, phenyloin, pilsicamide, propafenone, flecamide, atenolol, acebutolol, sotalol, propranolol, metoprolol, pindolol, amiodarone, nifekalant, diltiazem, bepridil, moricizine, tocamide, encamide, propafenone, esmolol, artilide, bretylium, clofilium, isobutilide, sotalol, azimilide, dofetilide, dronedarone, ersentilide, ibutilide, tedisamil, trecetilide, digitalis, adenosine, nickel chloride, and magnesium ions and verapamil.

Examples of the antihyperlipidemic drug include atorvastatin, simvastatin, pravastatin sodium, fluvastatin sodium, clinofibrate, clofibrate, simfibrate, fenofibrate, bezafibrate, colestimide, colestyramine, mevastatin ((2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), fluvastatin ((3R,5S,6E)-rel-7-[3-(4-fluorophenyl)-1-(1-methylethyl)-1H-indol-2-yl]-3,5-dihydroxy-6-heptenoic acid), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin ((βR,δR,1 S,2S,6S,8S,8aR)-1,2,6,7,8,8a-hexahydro-β,β,6-trihydroxy-2-methyl-8-[(2S)-2-methyl-1-oxobutoxy]-1-naphthaleneheptanoic acid), rosuvastatin ((3R,5 S,6E)-7-[4-(4-fluorophenyl)-6-(1-methylethyl)-2-[methyl(methylsulfonyl)amino]-5-pyrimidinyl]-3,5-dihydroxy-6-heptenoic acid), eptastatin, pitavastatin ((3R,5S,6E)-7-[2-cyclopropyl-4-(4-fluorophenyl)-3-quinolinyl]-3,5-dihydroxy-6-heptenoic acid), cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid), berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-, 2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-;4-(4-fluorophenyl)-2-(1-methylethyl)-3-[2-(tetrahydro-4-hydroxy-6-oxo-2H-pyran-2-yl)ethenyl]-1(2H)-isoquinolinone), SDZ-265859, BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5 S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,4R)-tetrahydro-4-hydroxy-6-oxo-2H-pyran-2-yl]ethyl]-1-naphthalenyl ester), and L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester).

Examples of the immunosuppressant include azathioprine, mizoribine, cyclosporine, tacrolimus, gusperimus, and methotrexate.

It should be understood that the methods of treatment of cardiovascular disease according to this invention may include conjointly administering one or more of the above agents either as a separate dosage form or as part of a composition that also comprises a statin, a compound of formula A, a compound of any one of formulae 1 to 44, lipoxin compound, an oxylipin compound, aspirin and/or an omega-3 fatty acid, and optionally further comprising a statin. Moreover, the use of a composition comprising both a statin and a compound of formula A, a compound of any one of formulae 1 to 44, a lipoxin compound, an oxylipin compound, or aspirin and/or omega-3 fatty acid according to this invention in the treatment of cardiovascular disease, does not preclude the separate but conjoint administration of another statin.

The method of increasing serum HDL concentration, reducing serum LDL/HDL ratio or reducing total serum cholesterol concentration in a patient according to this invention may additionally comprise administering to said patient another active ingredient other than a statin. Such additional active ingredient may be selected from a non-statin cholesterol lowering reagent, such as bile acid sequestrants (colesevelam, cholestyramine and colestipol), niacin, fibrates (gemfibrozil, probucol and clofibrate).

In certain embodiments, different compounds of formula A, compounds of any one of formulae 1 to 44, lipoxin compounds, or oxylipin compounds may be conjointly administered with one another, and such combinations may be conjointly administered with other therapeutics as discussed above. In certain embodiments, different compounds of formula A, compounds of any one of formulae 1 to 44, lipoxin compounds, or oxylipin compounds may be conjointly administered with a combination of aspirin and an omega-3 fatty acid, and such combinations may be conjointly administered with other therapeutics as discussed above.

In embodiments where a combination of aspirin and an omega-3 fatty acid are administered, the aspirin and omega-3 fatty acid can be administered simultaneously, e.g., as a single formulation comprising both components or in separate formulations, or can be administered at separate times, provided that, at least at certain times during the therapeutic regimen, both the aspirin and omega-3 fatty acid are present simultaneously in the patient at levels that allow the omega-3 fatty acid to be metabolized as described in Serhan, et. al., 2002, J. Exp. Med., 196: 1025-1037. In certain such embodiments, the omega-3 fatty acid is provided in the form of a partially purified natural extract, such as fish oil, while in other embodiments, the omega-3 fatty acid may be provided as a substantially pure preparation of one or more omega-3 fatty acids, such as a C18:3, C20:5, or C22:6 fatty acid, particularly eicosapentaenoic acid or docosahexaenoic acid. A substantially pure preparation of one or more omega-3 fatty acids refers to a composition wherein the fatty acid component is at least 90%, at least 95%, or even at least 98% of one or more omega-3 fatty acids, such as one or more specified omega-3 fatty acids. Non-fatty acid components, such as excipients or other materials added during formulation, are not considered for the purpose of determining whether the fatty acid component meets the desired level of purity.

In certain embodiments, a COX-2 inhibitor other than aspirin, such as celecoxib, rofecoxib, valdecoxib, lumiracoxib, etoricoxib, NS-398, or parecoxib, may be used in combination with an omega-3 fatty acid for the treatment of cardiovascular disease in any of the various embodiments discussed herein. The combination of different COX-2 inhibitors with an omega-3 fatty acid may result in the production of different subsets or proportions of active omega-3 metabolites.

This invention includes the use of pharmaceutically acceptable salts of compounds of formula A, compounds of any one of formulae 1 to 44, lipoxin compounds, or oxylipin compounds and/or statins in the compositions and methods of the present invention. In certain embodiments, contemplated salts of the invention include alkyl, dialkyl, trialkyl or tetra-alkyl ammonium salts. In certain embodiments, contemplated salts of the invention include Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The biological activity, such as anti-inflammatory activity, of a compound of formula A, a compound of any one of formulae 1 to 44, a lipoxin compound, an oxylipin compound, combination of aspirin and an omega-3 fatty acid, or statin can be assessed using techniques well known in the art, such as those discussed below.

Assay for Anti-Inflammatory Effect

Human endothelial cells or human leukocytes (e.g., monocytes, lymphocytes, and neutrophils), separately or in combination, are subjected in vitro to one or more proinflammatory and/or proliferative stimuli and secreted mediators of inflammation, such as cytokines, chemokines, and/or components involved in intracellular kinase pathways involved in their formation, are measured. Differences in these measurements between control cells and cells preincubated with a test anti-inflammatory composition, such as a composition comprising a compound of formula A, a compound of any one of formulae 1 to 44, a lipoxin compound, or an oxylipin compound and a statin, in inhibiting the formation of these mediators can be determined over different time courses and/or using a wide range of concentrations of the test composition.

Models for Determining the Therapeutic Effect of a Compound of Formula A, a Compound of Any One of Formulae 1 to 44, a Lipoxin Compound, an Oxylipin Compound, or a Combination of Aspirin and an Omega-3 Fatty Acid with or without a Statin on Lipid (LDL)-Dependent Vascular Pathology

Lipid depositions in the aorta can be quantified in rabbits made hypercholesterolemic by feeding them a high-fat diet, and differences in treated and control animals can be determined.

Development of atherosclerotic lesions in mice with LDL-receptor deficiency (ApoE −/−) show increased levels of cholesterol (LDL) can be quantified, and results in treated and control animals compared.

Polxamer 407 can be used to elevate LDL and triglycerides in C57BL/6 mice, and these levels can be monitored in the presence and absence of a test treatment regimen to investigate the treatment's effects on lipid metabolism.

In all three models, statins and compounds of formula A, compounds of any one of formulae 1 to 44, lipoxin compounds, oxylipin compounds, or a combination of aspirin and an omega-3 fatty acid separately and/or in combination can be tested for the ability to prevent formation of lipid dependent plaques in vessel walls, particularly the aorta, in a dose-dependent manner.

### INCORPORATION BY REFERENCE

All publications and patents mentioned herein are hereby incorporated by reference in their entirety as if each individual publication or patent was specifically and individually indicated to be incorporated by reference. In particular, compounds disclosed in the following patents and applications are incorporated by reference as suitable for use in methods of the present invention: US 2003/0191184, WO 2004/014835, WO 2004/078143, U.S. Pat. No. 6,670,396, US 2003/0236423, US 2005/0228047, US 2002/0107289, US 2004/0019110, US 2006/0009521, US 2005/0203184, US 2005/0113443, WO2006/055965. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating cardiovascular disease in a patient comprising administering to said patient a compound of formula A, a compound of any one of formulae 1 to 44, a lipoxin compound, or an oxylipin compound; and administering to said patient a statin conjointly with said compound of formula A, compound of any one of formulae 1 to 44, lipoxin compound, or oxylipin compound.

2. The method according to claim 1, further comprising administering to the patient a cardiovascular agent other than the compound of formula A, compound of any one of formulae 1 to 44, lipoxin compound, or oxylipin compound and the statin.

3. A method of increasing serum HDL, or decreasing serum LDL/HDL ratio or decreasing total serum cholesterol in a patient in need thereof comprising administering to said patient a compound of formula A, a compound of any one of formulae 1 to 44, a lipoxin compound, or an oxylipin compound.

4. The method according to claim 3, further comprising administering to the patient a cholesterol-lowering compound.

5. The method according to claim 4, wherein the cholesterol-lowering compound is a statin.

6. The method according to claim 5, wherein the amount of cholesterol-lowering compound administered to the patient is less than the amount that achieves a therapeutic effect when used in a monotherapy in the absence of the compound of formula A, compound of any one of formulae 1 to 44, lipoxin compound, or oxylipin compound.

7. The method according to claim 3, wherein the amount of compound of formula A, compound of any one of formulae 1 to 44, lipoxin compound, or oxylipin compound administered to the patient is less than an amount that achieves an anti-inflammatory effect in the absence of the cholesterol-lowering compound.

8. A method of reducing the dose of a statin required to increase serum HDL, decrease serum LDL/HDL ratio, or decrease serum cholesterol in a patient, comprising conjointly administering to the patient a compound of formula A, a compound of any one of formulae 1 to 44, a lipoxin compound, or an oxylipin compound and the statin.

9. A pyrogen-free composition comprising:
a compound of formula A, a compound of any one of formulae 1 to 44, a lipoxin compound, or an oxylipin compound;
a statin; and
a pharmaceutically acceptable carrier.

10. The composition according to claim 9, further comprising a cardiovascular agent other than a statin or a compound of formula A, a compound of any one of formulae 1 to 44, a lipoxin compound, or an oxylipin compound.

11. The method according to any one of claims 1 to 8, wherein the compound of formula A, compound of any one of formulae 1 to 44, lipoxin compound, or oxylipin compound is selected from a compound of any one of Formulae 1 to 44 or 50 to 115.

12. The composition according to claim 9 or 10, wherein the compound of formula A, compound of any one of formulae 1 to 44, lipoxin compound, or oxylipin compound is selected from a compound of any one of Formulae 1 to 44 or 50 to 115.

13. The method according to any one of claims 1, 2, 5, 6, or 8, wherein the statin is selected from a compound of any one of Formulae 200 to 207.

14. The composition according to claim 9 or 10, wherein the statin is selected from a compound of any one of Formulae 200 to 207.

15. A method of treating cardiovascular disease in a patient, comprising conjointly administering to said patient aspirin, an omega-3 fatty acid, and a statin.

16. The method according to claim 15, further comprising administering to the patient a cardiovascular agent other than aspirin and the statin.

17. A method of increasing serum HDL, or decreasing serum LDL/HDL ratio, or decreasing total serum cholesterol in a patient in need thereof, comprising administering to said patient a combination of aspirin and an omega-3 fatty acid.

18. The method according to claim 17, further comprising administering to the patient a cholesterol-lowering compound.

19. The method according to claim 18, wherein the cholesterol-lowering compound is a statin.

20. The method according to claim 19, wherein the amount of cholesterol-lowering compound administered to the patient is less than the amount that achieves a therapeutic effect when used in a monotherapy in the absence of the combination of aspirin and an omega-3 fatty acid.

21. The method according to claim 18, wherein the amount of the combination of aspirin and an omega-3 fatty acid administered to the patient is less than an amount that achieves an anti-inflammatory effect in the absence of the cholesterol-lowering compound.

22. A method of reducing the dose of a statin required to increase serum HDL, decrease serum LDL/HDL ratio, or decrease serum cholesterol in a patient, comprising conjointly administering to the patient aspirin, an omega-3 fatty acid, and the statin.

23. A pyrogen-free composition comprising aspirin, an omega-3 fatty acid, a statin, and a pharmaceutically acceptable carrier.

24. The composition according to claim 23, further comprising a cardiovascular agent other than the statin or aspirin.

25. The method according to any one of claims 15, 16, 19, 20, or 22, wherein the statin is selected from a compound of any one of Formulae 200 to 207.

26. The composition according to claim 23 or 24, wherein the statin is selected from a compound of any one of Formulae 200 to 207.
